SEQUENCE LISTING (48 sequences)

SEQ ID NO: 1 - DNA, 282 bp, Artificial Sequence
  Synthetic construct
acttcaatttactatgtagcaaaggatactccgacgcggccgcagcatatagcctggtggttcaggcggcgcatttttattgctgtgttgcgctgtaattcttctatttctgatgctgaatcaatgatgtctgccatctttcattaatccctgaactgttggttaatacgcttgagggtgaatgcgaataataaaaaaggagcctgtagctccctgatgattttgcttttcatgttcatcgttccttaaaagacgcagtttaacactggccgtcgttttaca

SEQ ID NO: 2 - DNA, 252 bp, Artificial Sequence
  Synthetic construct
taaaacgacggccagtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccaggctgcggccgcgtcggagtatcctttgctacatagtaaattgaagt

SEQ ID NO: 3 - DNA, 279 bp, Artificial Sequence
  Synthetic construct
gtaaaacgacggccagtctgcgattctcaccaataaaaaacgcccggcggcaaccgagcgttctgaacaaatccagatggagttctgaggtcattactggatctatcaacaggagtcattatgacaaatacagcaaaaatactcaacttcggcagaggtaactttgccggacaggagcgtaatgtggcagatctcgatgatggttacgccagactatcaaatatgctgcttgagctgcggccgcgtcggagtatcctttgctacatagtaaattgaagt

SEQ ID NO: 4 - DNA, 11 bp, Artificial Sequence
  Synthetic construct
cgcacttcaat

SEQ ID NO: 5 - DNA, 10 bp, Artificial Sequence
  Synthetic construct
ggtagcaact

SEQ ID NO: 6 - DNA, 50 bp, Artificial Sequence
  Synthetic construct
ccgtcggtcccaaactacaccccaacctgcatacctcgacggtagcaact

SEQ ID NO: 7 - DNA, 48 bp, Artificial Sequence
  Synthetic construct
gcgagccccacccacccacccacccacccacccagctcgcacttcaat

SEQ ID NO: 8 - DNA, 44 bp, Artificial Sequence
  Synthetic construct
ccgtcgtaggaaacaccaaagatgatatttcgacggtagcaact

SEQ ID NO: 9 - DNA, 43 bp, Artificial Sequence
  Synthetic construct
gcgagccagttacattctcccagttgattgctcgcacttcaat

SEQ ID NO: 10 - DNA, 25 bp, Artificial Sequence
  Synthetic construct
tagcaacugtaaaacgacggccagt

SEQ ID NO: 11 - DNA, 25 bp, Artificial Sequence
  Synthetic construct
acttcaauttactatgtagcaaagg

SEQ ID NO: 12 - DNA, 50 bp, Artificial Sequence; identical to SEQ ID NO: 6
  Synthetic construct

SEQ ID NO: 13 - DNA, 48 bp, Artificial Sequence; identical to SEQ ID NO: 7
  Synthetic construct

SEQ ID NO: 14 - DNA, 34 bp, Artificial Sequence
  Synthetic construct
agccccacccacccacccacccacccacccagct

SEQ ID NO: 15 - DNA, 8 bp, Artificial Sequence
  Synthetic construct
tagcaact

SEQ ID NO: 16 - DNA, 8 bp, Artificial Sequence
  Synthetic construct
acttcaat

SEQ ID NO: 17 - DNA, 29 bp, Artificial Sequence
  Synthetic construct
aggtatgcaggttggggtgtagtttggga

SEQ ID NO: 18 - DNA, 38 bp, Artificial Sequence
  Synthetic construct
gtcggtcccaaactacaccccaacctgcatacctcgac

SEQ ID NO: 19 - DNA, 51 bp, Artificial Sequence
  Synthetic construct
cgcacttcaatttactatgtagcaaaggatactccgacgcggccgccgccc

SEQ ID NO: 20 - DNA, 51 bp, Artificial Sequence
  Synthetic construct
cgcacttcaatttactatgtagcaaaggatactccgacgcggccgcgaccc

SEQ ID NO: 21 - DNA, 51 bp, Artificial Sequence
  Synthetic construct
cgcacttcaatttactatgtagcaaaggatactccgacgcggccccagctc

SEQ ID NO: 22 - DNA, 51 bp, Artificial Sequence
  Synthetic construct
cgcacttcaatttactatgtagcaaaggatactccgacgcggccccatctc

SEQ ID NO: 23 - DNA, 51 bp, Artificial Sequence
  Synthetic construct
cgcacttcaatttactatgtagcaaaggatactccgacgcggccccgtccc

SEQ ID NO: 24 - DNA, 52 bp, Artificial Sequence
  Synthetic construct
cgcacttcaatttactatgtagcaaaggatactccgaccccgccgctgcccc

SEQ ID NO: 25 - DNA, 51 bp, Artificial Sequence
  Synthetic construct
cgcacttcaatttactatgtagcaaaggatactccgacccgcccccccctg

SEQ ID NO: 26 - DNA, 51 bp, Artificial Sequence
  Synthetic construct
cgcacttcaatttactatgtagcaaaggatactccgacccggccccacccc

SEQ ID NO: 27 - DNA, 51 bp, Artificial Sequence
  Synthetic construct
cgcacttcaatttactatgtagcaaaggatactccgacccggcccccccct

SEQ ID NO: 28 - DNA, 51 bp, Artificial Sequence
  Synthetic construct
cgcacttcaatttactatgtagcaaaggatactccgacccggccccgtcct

SEQ ID NO: 29 - DNA, 51 bp, Artificial Sequence
  Synthetic construct
cgcacttcaatttactatgtagcaaaggatactccacccccgccaccgcct

SEQ ID NO: 30 - DNA, 51 bp, Artificial Sequence
  Synthetic construct
cgcacttcaatttactatgtagcaaaggatactccacccccgccctctccc

SEQ ID NO: 31 - DNA, 51 bp, Artificial Sequence
  Synthetic construct
cgcacttcaatttactatgtagcaaaggatactcccaccccccctcctccc

SEQ ID NO: 32 - DNA, 51 bp, Artificial Sequence
  Synthetic construct
ggtagcaactgtaaaacgacggccagtctgcgattctcaccaataaaaaac

SEQ ID NO: 33 - DNA, 51 bp, Artificial Sequence
  Synthetic construct
ggtagcaactgtaaaacgacggccagtctgcgattctccccaataaaaaac

SEQ ID NO: 34 - DNA, 51 bp, Artificial Sequence
  Synthetic construct
ggtagcaactgtaaaacgacggccagtctgcgattctcaccaataaaaacc

SEQ ID NO: 35 - DNA, 51 bp, Artificial Sequence
  Synthetic construct
ggtagcaactgtaaaacgacggccagtctgcgattctcacccataaaaaac

SEQ ID NO: 36 - DNA, 51 bp, Artificial Sequence
  Synthetic construct
ggtagcaactgtaaaacgacggccagtctgcgattctcaccaataaaaaaa

SEQ ID NO: 37 - DNA, 51 bp, Artificial Sequence
  Synthetic construct
ggtagcaactgtaaaacgacggccagtctgcgattctcaccaatacaaaac

SEQ ID NO: 38 - DNA, 51 bp, Artificial Sequence
  Synthetic construct
ggtagcaactgtaaaacgacggccagtgaatgcaaagaagataaccgcttc

SEQ ID NO: 39 - DNA, 51 bp, Artificial Sequence
  Synthetic construct
ggtagcaactgtaaaacgacggccagtgaatgcaaagaagataaccccttc

SEQ ID NO: 40 - DNA, 51 bp, Artificial Sequence
  Synthetic construct
ggtagcaactgtaaaacgacggccagtgaatgcaaagaagataaccgctcc

SEQ ID NO: 41 - DNA, 51 bp, Artificial Sequence
  Synthetic construct
ggtagcaactgtaaaacgacggccagtgaatgcaaagaagataacccctcc

SEQ ID NO: 42 - DNA, 51 bp, Artificial Sequence
  Synthetic construct
ggtagcaactgtaaaacgacggccagtgaatgcaaagaagataaccgcctc

SEQ ID NO: 43 - DNA, 51 bp, Artificial Sequence
  Synthetic construct
ggtagcaactgtaaaacgacggccagtgttaaactgcgtctttatggaacg

SEQ ID NO: 44 - DNA, 51 bp, Artificial Sequence
  Synthetic construct
ggtagcaactgtaaaacgacggccagtgttaaataagcaaatattcgaaac

SEQ ID NO: 45 - DNA, 51 bp, Artificial Sequence
  Synthetic construct
ggtagcaactgtaaaacgacggccagtgttaaacttcgtctttatcgatct

SEQ ID NO: 46 - DNA, 51 bp, Artificial Sequence
  Synthetic construct
ggtagcaactgtaaaacgacggccagtgttaaactgcgtctttaatgaacg

SEQ ID NO: 47 - DNA, 51 bp, Artificial Sequence
  Synthetic construct
ggtagcaactgtaaaacgacggccagtgttaaactgcgcctttaattaacg

SEQ ID NO: 48 - DNA, 51 bp, Artificial Sequence
  Synthetic construct
ggtagcaactgtaaaacgacggccagtgttaaaaaagaagattattccttc

(19) United States
(12) Patent Application Publication
(10) Pub. No.: US 2021/0189460 A1
(43) Pub. Date: Jun. 24, 2021

(54) SEQUENTIAL PAIRED-END SEQUENCING

(71) Applicant: QIAGEN Sciences LLC, Germantown, MD (US)

(72) Inventors: Seiyu Hosono, Arlington, MA (US); Robert Pinard, Andover, MA (US)

(21) Appl. No.: 17/050,052

(22) PCT Filed: Apr. 24, 2019
(86) PCT No.: PCT/US19/28908

§ 371 Date: Oct. 23, 2020

Related U.S. Application Data
(60) Provisional application No. 62/662,511, filed on Apr. 25, 2018.

(51) Int. Cl. C12Q 1/6806; C12Q 1/6869; C12N 15/113

(52) U.S. Cl. CPC C12Q 1/6806; C12Q 1/6869; C12N 15/113; C12N 2310/532; C12N 2310/346; C12N 2310/51; C12N 2310/344; C12N 2310/531

(57) ABSTRACT

Disclosed are compositions and methods for determining the nucleotide sequence of sequences of interest using paired-end sequencing. Dumbell circular templates can be generated and used in a rolling circle amplification reaction by ligating two hairpin adaptors on a double-stranded amplicon. Disclosed also are methods using double-stranded DNA, including both sense and antisense strands in a single circle to sequence, sequentially from the same concatemers.

### CROSS REFERENCE TO RELATED APPLICATIONS

This application claims the benefit of the filing date of U.S. Provisional Application No. 62/662,511, which was filed on Apr. 25, 2018. The content of this earlier filed application is hereby incorporated by reference herein in its entirety.

### SEQUENCE LISTING

The sequence listing submitted herewith as a text filed named “17104_0060P1_SL.txt,” created on Apr. 23, 2019, and having a size of 12,288 bytes is herein incorporated by reference in its entirety pursuant to 37 C.F.R. § 1.52(e)(5).

### BACKGROUND

High-throughput sequence analysis of genomic DNA is important to glean biological information related to health and disease of humans, plants and animals. Many currently available approaches are capable of parallel analysis of target sequences, but are not without limitations, including, for example, the determination of a few nucelotides and reducing the experimental efficiency. A need exists for a sequencing technique that permits an efficient determination of long-sequence read-lengths in a large-scale setting in which both the sense and anti-sense strands of a polynucleotide can be sequenced in the same trace.

### SUMMARY

Disclosed herein are compositions and methods useful in a sequential paired-end approach to sequencing.

Disclosed herein are methods of preparing a plurality of concatamers.

Disclosed herein are methods of preparing a plurality of concatamers comprising: forming a first partially double stranded circular DNA wherein the partially double stranded circular DNA contains (i) a sequence of interest having a first strand and a second strand, (ii) a first hairpin adaptor, and (iii) a second hairpin adaptor, wherein the first and second strands of the sequence of interest are complementary to each other and wherein the first hairpin adaptor comprises a first primer binding site and the second hairpin adaptor comprises a second primer binding site; and amplifying the first partially double stranded circular DNA via rolling circle amplification, wherein amplification of the first partially double stranded circular DNA results in replicated strands, wherein during amplification at least one of the replicated strands is displaced from the first partially double stranded circular DNA by strand displacement replication;

wherein the amplification of the first partially double stranded circular DNA results in a plurality of concatemers.

Disclosed herein are methods of preparing a plurality of concatamers comprising: forming a first partially double stranded circular DNA wherein the partially double stranded circular DNA contains (i) a sequence of interest having a first strand and a second strand, (ii) a first hairpin adaptor, and (iii) a second hairpin adaptor, wherein the first and second strands of the sequence of interest are complementary to each other and wherein the first hairpin adaptor comprises a first primer binding site and the second hairpin adaptor comprises a second primer binding site; and amplifying the first partially double stranded circular DNA via rolling circle amplification, wherein amplification of the first partially double stranded circular DNA results in replicated strands, wherein during amplification at least one of the replicated strands is displaced from the first partially double stranded circular DNA by strand displacement replication;

wherein the amplification of the first partially double stranded circular DNA results in a plurality of concatemers, wherein the first partially double-stranded circular DNA is formed by: (i) contacting a target nucleic acid molecule that contains a sequence of interest, with a first primer set, wherein at least one primer in the first primer set comprises a uracil at the 3′ end; (ii.) incubating the target nucleic acid molecule with the first primer set under conditions that promote hybridization and replication of the target nucleic acid molecule, thereby producing a double-stranded amplicon, wherein the double-stranded amplicon comprises a first and second strand; (iii.) contacting the double-stranded amplicon with an enzyme to produce a first terminal overhang at the 3′ end of the first strand and a second terminal overhang at the 3′ end of the second strand of the double-stranded amplicon; (iv.) contacting the double-stranded amplicon of (iii) with the first hairpin adaptor and the second hairpin adaptor, wherein the first hairpin adaptor comprises a first sequence complementary to the first terminal overhang of the double-stranded amplicon and the second hairpin adapter comprises a second sequence complementary to the second terminal overhang; (v.) incubating the double-stranded amplicon with the first hairpin adaptor and the second hairpin adapter under conditions that promote hybridization between the first hairpin adapter with the first terminal overhang of the double-stranded amplicon and the second hairpin adapter with the second terminal overhang of the double-stranded amplicon; and (vi.) ligating the first hairpin adaptor to the first terminal overhang and the second hairpin adaptor with the second terminal overhang thereby forming the first partially double stranded circular DNA.

Disclosed herein are methods of preparing a plurality of concatamers comprising: forming a first partially double stranded circular DNA wherein the partially double stranded circular DNA contains (i) a sequence of interest having a first strand and a second strand, (ii) a first hairpin adaptor, and (iii) a second hairpin adaptor, wherein the first and second strands of the sequence of interest are complementary to each other and wherein the first hairpin adaptor comprises a first primer binding site and the second hairpin adaptor comprises a second primer binding site; and amplifying the first partially double stranded circular DNA via rolling circle amplification, wherein amplification of the first partially double stranded circular DNA results in replicated strands, wherein during amplification at least one of the replicated strands is displaced from the first partially double stranded circular DNA by strand displacement replication; wherein the amplification of the first partially double stranded circular DNA results in a plurality of concatemers, wherein the first partially double-stranded circular DNA is formed by: (i.) contacting a target nucleic acid molecule that contains a sequence of interest, with a first primer set, wherein at least one primer in the first primer set comprises a uracil at the 3′ end; (ii.) incubating the target nucleic acid molecule with the first primer set under conditions that promote hybridization and replication of the target nucleic acid molecule, thereby producing a double-stranded amplicon, wherein the double-stranded amplicon comprises a first and second strand; (iii.) contacting the double-stranded amplicon with an enzyme to produce a first terminal overhang at the 3′ end of the first strand and a second terminal overhang at the 3′ end of the second strand of the double-stranded amplicon; (iv.) contacting the double-stranded amplicon of iii) with the first hairpin adaptor and the second hairpin adaptor, wherein the first hairpin adaptor comprises a first sequence complementary to the first terminal overhang the double-stranded amplicon and the second hairpin adapter comprises a second sequence complementary to the second terminal overhang at the 3′ end of the double-stranded amplicon; (v.) incubating the double-stranded amplicon with the first hairpin adaptor and second hairpin adapter under conditions that promote hybridization between the first hairpin adapter with the first terminal overhang of the double-stranded amplicon and the second hairpin adapter with the second terminal overhang of the double-stranded amplicon; and ligating the first hairpin adaptor to the first terminal overhang and the second hairpin adaptor with the second terminal overhang thereby forming a closed circular template, wherein the closed circular template is in a dumbbell configuration having a double-stranded portion and two single stranded portions.

Disclosed herein are methods of preparing a plurality of concatamers comprising: forming a first partially double stranded circular DNA wherein the partially double stranded circular DNA contains (i) a sequence of interest having a first strand and a second strand, (ii) a first hairpin adaptor, and (iii) a second hairpin adaptor, wherein the first and second strands of the sequence of interest are complementary to each other and wherein the first hairpin adaptor comprises a first primer binding site and the second hairpin adaptor comprises a second primer binding site; and amplifying the first partially double stranded circular DNA via rolling circle amplification, wherein amplification of the first partially double stranded circular DNA results in replicated strands, wherein during amplification at least one of the replicated strands is displaced from the first partially double stranded circular DNA by strand displacement replication;

wherein the amplification of the first partially double stranded circular DNA results in a plurality of concatemers, wherein the amplification of the first partially double-stranded circular DNA comprises: (i.) bringing into contact the partially double stranded circular DNA, a DNA polymerase, and a second primer set, wherein the primers of the second primer set hybridizes to the single-stranded portions of the partially double stranded circular DNA; and (ii.) incubating the partially double stranded circular DNA under conditions that promote replication of the partially double stranded circular DNA;
wherein the replication of the partially double stranded circular DNA results in replicated strands, wherein during replication at least one of the replicated strands is displaced from the partially double stranded circular DNA by strand displacement replication of another replicated strand.

Also disclosed are methods of identifying at least one nucleotide of a sequence of interest, the method comprising: (a) forming a first partially double stranded circular DNA wherein the partially double stranded circular DNA contains (i) a sequence of interest, (ii) a first hairpin adaptor, and (iii) a second hairpin adaptor, wherein the first hairpin adaptor comprises a first primer binding site and the second hairpin adaptor comprises a second primer binding site; (b) amplifying the first partially double stranded circular DNA via rolling circle amplification, wherein amplification of the first partially double stranded circular DNA results in replicated strands, wherein during amplification at least one of the replicated strands is displaced from the first partially double stranded circular DNA by strand displacement replication, wherein the amplification of the first partially double stranded circular DNA results in a plurality of partially double stranded concatamers; (c) contacting at least one of the partially double stranded concatamers with a first primer, wherein the first primer hybridizes to the first or second primer binding, and (d) identifying at least one nucleotide of the sequence of interest adjacent or close to the first primer binding site

Also disclosed are methods of identifying at least one nucleotide of a sequence of interest, the method comprising: (a) forming a first partially double stranded circular DNA wherein the partially double stranded circular DNA contains (i) a sequence of interest, (ii) a first hairpin adaptor, and (iii) a second hairpin adaptor, wherein the first hairpin adaptor comprises a first primer binding site and the second hairpin adaptor comprises a second primer binding site; (b) amplifying the first partially double stranded circular DNA via rolling circle amplification, wherein amplification of the first partially double stranded circular DNA results in replicated strands, wherein during amplification at least one of the replicated strands is displaced from the first partially double stranded circular DNA by strand displacement replication, wherein the amplification of the first partially double stranded circular DNA results in a plurality of partially double stranded concatamers, (c) contacting at least one of the partially double stranded concatamers with a first primer, wherein the first primer hybridizes to the first primer binding site, (d) extending the first primer in the presence of one or more dideoxynucleotides thereby generating a first primer elongation product, (e) contacting at least one of the partially double stranded concatamers with a second primer, wherein the second primer hybridizes to the second primer binding site; (f) extending the second primer, thereby generating a second primer elongation product; and (g) identifying at least one nucleotide of the sequence of interest adjacent or close to the first primer binding site and at least one nucleotide of the sequence of interest adjacent or close to the second primer binding site.

Also disclosed are arrays for identifying at least one nucleotide of a sequence of interest, comprising a plurality of amplicons immobilized on a surface, wherein each of the amplicons comprise two or more concatemers, wherein each of the concatemers comprises: a first hairpin adapter, at least one sequence of interest and a second hairpin adapter formed by a process comprising: (a) contacting a target nucleic acid molecule comprising the sequence of interest, with a first primer set having a uracil at the 3′ end, wherein the target nucleic acid molecule is double-stranded; incubating the target nucleic acid molecule with a first primer set under conditions that promote hybridization and replication of the target nucleic acid molecule, thereby producing a double-stranded template, wherein the double-stranded template comprises a first and second strand; (b) contacting the double-stranded template with an enzyme to produce a first terminal overhang and a second terminal overhang at the 3′ end of the first and second strand of the double-stranded template; (c) contacting the double-stranded template with the first hairpin adaptor and second hairpin adaptor, wherein the first hairpin adaptor comprises a first sequence complementary to the first terminal overhang the double-stranded template and the second hairpin adapter comprises a second sequence complementary to the second terminal overhang at the 3′ end of the double-stranded template; (d) incubating the double-stranded template with the first hairpin adaptor and second hairpin adapter under conditions that promote hybridization between the first hairpin adapter with the first terminal overhang of the double-stranded template and the second hairpin adapter with the second terminal overhang of the double-stranded template; and (e) ligating the first hairpin adaptor to the first terminal overhang and the second hairpin adaptor with the second terminal overhang forming a partially double stranded circular DNA; wherein the first adaptor is different from the second adaptor and comprises a first restriction enzyme binding site and the second adaptor comprises a second restriction enzyme binding site, wherein the first and second restriction enzyme binding sites that cleaves DNA a t cleavage site.

Disclosed herein are combinations configured for identifying at least one nucleotide of a sequence of interest, comprising: (a) providing an array for identifying at least one nucleotide of a sequence of interest, the array comprising a plurality of amplicons having been formed and immobilized on a surface.

### BRIEF DESCRIPTION OF THE DRAWINGS

FIG. 1 is a schematic showing DNA segments of interest during polymerase chain reaction (PCR) amplification using uracil-containing primers.

FIG. 2 is a schematic illustrating the formation of a closed circular template (e.g., dumbbell circle).

FIG. 3 is a schematic showing the formation of concatemers via rolling circle amplification using a closed circular template.

FIG. 4 is a schematic depicting sequential sequencing using two primer strands.

FIG. 5 is a schematic showing the sequencing of strand A followed by a one-base addition-blocking step using dideoxynucleotides.

FIG. 6 is a schematic illustrating the sequencing of strand B.

FIG. 7 shows the results of PCR, User™ treatment and hairpin ligation.

FIG. 8 shows a sequencing image (top panel) using first strand A and second strand B (bottom panel). Sequences: CGCACTTCAAT (SEQ ID NO: 18); and GGTAGCAACT (SEQ ID NO: 19).

FIG. 9 shows an example of the sequence occurrences obtained.

FIG. 10 shows the results of sequential paired-end sequencing.

FIG. 11 shows that the amount of reads mapped to the lambda clone sequences and paired physically.

FIG. 12 shows the number of reads that were mapped to one strand (red) or to both strands (blue) at the same time and to a unique location.

FIG. 13 shows the PCR primer products using touchdown PCR.

### DETAILED DESCRIPTION OF THE INVENTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components. The term sample can also refer to a “cancer sample” or “sample of the cancer” or the like. The sample can be obtained via biopsy such as needle biopsy, surgical biopsy, etc. A cancer sample includes, for example, a specimen of cancers, parts of a cancer, cancer cells derived from a cancer (including cancer cell lines derived from a cancer and are grown in cell culture) and also the cancer mass as a whole, cell lines, cells and/or tissue derived from a subject that are suspected of being cancerous or suspected of comprising cancerous cells. Thus, it is possible that the cancer sample may also comprise non-cancerous cells.

As used herein, the term “comprising” can include the aspects “consisting of” and “consisting essentially of” “Comprising” can also mean “including but not limited to.”

The term “double-stranded amplicon” or “amplicon” is used herein to refer to an elongation product.

The phrase “at least” preceding a series of elements is to be understood to refer to every element in the series. For example, “at least one” includes one, two, three, four or more.

As used herein, the term “target nucleic acid molecule,” “target sequence,” “target nucleic acid,” or “target polynucleotide” and the like are nucleic acids. A target nucleic acid molecule can be a portion of a gene, a regulatory sequence, genomic DNA, cDNA, RNA including mRNA and rRNA, or others. As described herein, the target nucleic acid molecule can be a target nucleic acid molecule from a sample, or a secondary target such as a product of an amplification reaction, etc. It may be any length.

The term “nucleic acids” or “oligonucleotide” or grammatical equivalents herein refers to at least two nucleotides covlanetly linked together. Nucleic acids containing one or more carbocyclic sugars are also included within the definition of nucleic acids. A “nucleic acid” will generally contain phosphodiester bonds, although in some cases (for example, in the construction of primers and probes, such as label probes), nucleic acid analogs are included that may have alternate backbones, comprising, for example, phosphoramide (Beaucage et al., Tetrahedron 49(10): 1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with bicyclic structures including locked nucleic acids, Koshkin et al., J. Am. Chem. Soc. 120:13252 3 (1998); positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169 176). Several nucleic acid analogs are described in Rawls, C & E News Jun. 2, 1997 page 35. All of these references are hereby expressly incorporated by reference. These modifications of the ribose-phosphate backbone may be done to increase the stability and half-life of such molecules in physiological environments. For example, PNA:DNA hybrids can exhibit higher stability and thus may be used in some embodiments.

Nucleic acids may be single-stranded or double-stranded, as specified, or contain portions of both double-stranded or single-stranded sequences. The nucleic acids may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

As used herein, the term “sequence of interest” is the object of amplification or sequencing and can be any nucleic acid. As used herein, the term “sequence of interest” can refer to a portion of a “target nucleic acid molecule,” “target sequence,” “target nucleic acid,” or “target polynucleotide” and the like. The sequence of interest can include multiple nucleic acid molecules, such as in the case of whole genome amplification, multiple sites in a nucleic acid molecule, or a single region of a nucleic acid molecule. A sequence of interest can be in any nucleic acid sample of interest and of any length. The term “sequence of interest” can also mean a nucleic acid sequence (e.g., a therapeutic gene), that is partly or entirely heterologous, i.e., foreign, to a cell into which it is introduced. The term “sequence of interest” can also mean a nucleic acid sequence that is partly or entirely homologous to an endogenous gene of the cell into which it is introduced. For example, a sequence of interest can be cDNA, DNA, or RNA including mRNA and rRNA or others. The term “sequence of interest” can also mean a nucleic acid sequence that is partly or entirely complementary to an endogenous gene of the cell into which it is introduced.

As used herein, “concatemers” refer to a form of target polynucleotides, containing multiple copies (e.g., monomers) of a target polynucleotide or a fragment of a target nucleotide. In some aspects, the concatamer contains a sequence of interest. A concatemer can be partially doube-stranded. In some aspects, the plurality of concatemers can serve as a target nucleic acid molecule for sequencing. In an aspect, the concatemers comprise a single-stranded DNA portion and a double-stranded DNA portion. In an aspect, the concatemers comprise a copy of the sequence of interest and a copy of at least one or both of the hairpin adapters. In an aspect, the concatemers comprise a copy of the sequence of interest and a copy of the first hairpin adapter and a copy of the second hairpin adapter. In some aspects, the concatemers disclosed herein comprise, in order, a portion of one of the hairpin adaptors, a copy of the first strand of the sequence of interest, a copy of one of the hairpin adaptors, and a copy of the second strand of the sequence of interest, wherein the first and second strands of the sequence of interest are hybridized together. In an aspect, the concatemers are immobilized on a surface.

As used herein, “hairpin adapter” refers to a nucleic acid that comprises a single stranded and a double stranded region. The double-stranded region is formed by hybridization between two separate regions of the hairpin adapter. A hairpin adapter comprises a hairpin loop.

As used herein, to cap an —OH group means to replace the “H” in the —OH group with a chemical group. As disclosed herein, the —OH group of a modified nucleotide can be capped or protected with a cleavable chemical group. To uncap or deprotect an —OH group means to cleave the chemical group from a capped or protected —OH group and to replace the chemical group with “H”, i.e., to replace the “R” in —OR with “H” wherein “R” is the chemical group used to cap or protect the —OH group.

The nucleotide bases are abbreviated as follows: adenine (A), cytosine (C), guanine (G), thymine (T), and uracil.

A nucleotide analogue or modified nucleotide refers to a chemical compound that is structurally and functionally similar to the nucleotide, i.e. the nucleotide analogue can be recognized by polymerase as a substrate. That is, for example, a nucleotide analogue comprising adenine or an analogue of adenine should form hydrogen bonds with thymine, a nucleotide analogue comprising C or an analogue of C should form hydrogen bonds with G, a nucleotide analogue comprising G or an analogue of G should form hydrogen bonds with C, and a nucleotide analogue comprising T or an analogue of T should form hydrogen bonds with A, in a double helix format.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, certain changes and modifications may be practiced within the scope of the appended claims.

### Compositions

### A. Target Nucleic Acid Molecule

A target nucleic acid molecule can be any nucleic acid. Target nucleic acid molecule can include multiple nucleic acid molecules, such as in the case of whole genome amplification, multiple sites in a nucleic acid molecule, or a single region of a nucleic acid molecule. For multiple strand displacement amplification, generally the target nucleic acid molecule is a single region in a nucleic acid molecule or nucleic acid sample. For whole genome amplification, the target nucleic acid molecule is the entire genome or nucleic acid sample.

A target nucleic acid molecule can be in any nucleic acid sample of interest. The source, identity, and preparation of many such nucleic acid samples are known. It is preferred that nucleic acid samples known or identified for use in amplification or detection methods be used for the method described herein. The nucleic acid sample can be, for example, a nucleic acid sample from one or more cells, tissue, or bodily fluids such as blood, urine, semen, lymphatic fluid, cerebrospinal fluid, or amniotic fluid, or other biological samples, such as tissue culture cells, buccal swabs, mouthwash, stool, tissues slices, biopsy aspiration, and archeological samples such as bone or mummified tissue. Target samples can be derived from any source including, but not limited to, eukaryotes, plants, animals, vertebrates, fish, mammals, humans, non-humans, bacteria, microbes, viruses, biological sources, serum, plasma, blood, urine, semen, lymphatic fluid, cerebrospinal fluid, amniotic fluid, biopsies, needle aspiration biopsies, cancers, tumors, tissues, cells, cell lysates, crude cell lysates, tissue lysates, tissue culture cells, buccal swabs, mouthwash, stool, mummified tissue, forensic sources, autopsies, archeological sources, infections, nosocomial infections, production sources, drug preparations, biological molecule productions, protein preparations, lipid preparations, carbohydrate preparations, inanimate objects, air, soil, sap, metal, fossils, excavated materials, and/or other terrestrial or extra-terrestrial materials and sources. The sample may also contain mixtures of material from one or more different sources. For example, nucleic acids of an infecting bacterium or virus can be amplified along with human nucleic acids when nucleic acids from such infected cells or tissues are amplified using the disclosed methods. Types of useful target samples include eukaryotic samples, plant samples, animal samples, vertebrate samples, fish samples, mammalian samples, human samples, non-human samples, bacterial samples, microbial samples, viral samples, biological samples, serum samples, plasma samples, blood samples, urine samples, semen samples, lymphatic fluid samples, cerebrospinal fluid samples, amniotic fluid samples, biopsy samples, needle aspiration biopsy samples, cancer samples, tumor samples, tissue samples, cell samples, cell lysate samples, crude cell lysate samples, tissue lysate samples, tissue culture cell samples, buccal swab samples, mouthwash samples, stool samples, mummified tissue samples, forensic samples, autopsy samples, archeological samples, infection samples, nosocomial infection samples, production samples, drug preparation samples, biological molecule production samples, protein preparation samples, lipid preparation samples, carbohydrate preparation samples, inanimate object samples, air samples, soil samples, sap samples, metal samples, fossil samples, excavated material samples, and/or other terrestrial or extra-terrestrial samples.

A target nucleic acid molecule can include damaged DNA and damaged DNA samples. For example, preparation of genomic DNA samples can result in damage to the genomic DNA (for example, degradation and fragmentation). This can make amplification of the genome or sequences in it both more difficult and provide less reliable results (by, for example, resulting in amplification of many partial and fragmented genomic sequences). Damaged DNA and damaged DNA samples are thus useful for the disclosed method of amplifying damaged DNA. Any degraded, fragmented or otherwise damaged DNA or sample containing such DNA can be used in the disclosed method.

The disclosed methods can involve the sequencing of nucleic acids (e.g., target nucleic acid molecules). In an aspect, the target nucleic acid molecules can be genomic DNA. The genomic DNA can be obtained using any technique known to one of ordinary skill in the art. Factors for isolating genomic DNA include, but are not limited to the following: 1) the DNA is free of DNA processing enzymes and contaminating salts; 2) the entire genome is equally represented; and 3) the DNA fragments are between about 5,000 and 100,000 bp in length.

In many cases, no digestion of the extracted DNA is required because shear forces created during lysis and extraction will generate fragments. In an aspect, shorter fragments (1-5 kb) can be generated by enzymatic fragmentation using restriction endonucleases. In an aspect, 10-100 genome-equivalents of DNA ensure that the population of fragments covers the entire genome. In some cases, it can be advantageous to provide carrier DNA, e.g. unrelated circular synthetic double-stranded DNA, to be mixed and used with the sample DNA whenever small amounts of sample DNA are available and there is danger of losses through nonspecific binding, e.g. to container walls and the like. In an aspect, the DNA is in a dumbbell formation, does not require denaturation. In an aspect, the DNA can be denatured after fragmentation to produce single-stranded fragments.

Target polynucleotides can be generated from a source nucleic acid, such as genomic DNA, by fragmentation to produce fragments of a specific size. In an aspect, the fragments can be 50 to 600 nucleotides in length. In another aspect, the fragments can be 300 to 600 or 200 to 2000 nucleotides in length. In another aspect, the fragments can be 10-100, 50-100, 50-300, 100-200, 200-300, 50-400, 100-400, 200-400, 400-500, 400-600, 500-600, 50-1000, 100-1000, 200-1000, 300-1000, 400-1000, 500-1000, 600-1000, 700-1000, 700-900, 700-800, 800-1000, 900-1000, 1500-2000, 1750-2000, and 50-2000 nucleotides in length. These fragments can in turn be circularized for use in a rolling circle amplification reaction or in other biochemical processes.

In an aspect, the target nucleic acid molecule can be double-stranded. By using double-stranded DNA, both sense (+) and antisense (−) strands can be used for pair-end sequencing. In an aspect, the target nucleic acid molecule comprises a sequence of interest. The sequence of interest can have a first strand and second strand. The first and second strands of the sequence of interest can be complementary to each other. In an aspect, the target nucleic acid molecule further comprises a first hairpin adapter and a second hairpin adapter.

In an aspect, the target nucleic acid molecule comprises an amplification region and a hybridization region. The hybridization region includes the sequences that can be complementary to the primers in a set of primers. The amplification region can be the portion of the amplification region that can be amplified. In an aspect, the amplification region can be downstream of or flanked by the hybridization region(s).

### B. Sequence of Interest

The disclosed methods involve utilizing DNA fragments comprising one or more sequences of interest from samples. A sample solution can comprise any number of components, including, but not limited to, bodily fluids (including, but not limited to, blood, urine, serum, lymph, saliva, anal and vaginal secretions, perspiration and semen) and cells of any organism; environmental samples (including, but not limited to, air, agricultural, water and soil samples); biological warfare agent samples; research samples (i.e. in the case of nucleic acids, the sample can be the products of an amplification reaction, including both target and signal amplification, such as PCR amplification reactions; purified samples, such as purified genomic DNA, RNA preparations, raw samples (bacteria, virus, genomic DNA)); and as will be appreciated by those skilled in the art, almost any experimental manipulation can be done on the samples. In an aspect, the samples can be mammalian samples. In an aspect, the samples can be human samples.

In general, cells from the target organism (e.g., animal, avian, mammalian) can be used. When genomic DNA is used, the amount of genomic DNA required for constructing arrays of the invention can vary. In an aspect, for mammalian-sized genomes, fragments can be generated from at least about 10 genome-equivalents of DNA. In another aspect, fragments can be generated from at least about 30 genome-equivalents of DNA. In another aspect, fragments can be generated from at least about 60 genome-equivalents of DNA.

The sequence of interest can be any nucleic acid sequence. In an aspect, the sequence of interest can be double-stranded. The hybridization region and the amplification region within the target nucleic acid molecule can be defined in terms of the relationship of the target nucleic acid molecule to the primers in a set of primers. The primers can be designed to match the chosen sequence of interest. In an aspect, the sequence to be amplified and the sites of hybridization of the primers can be separate since sequences in and around the sites where the primers hybridize can be amplified.

### C. Hairpin Adapter

Disclosed herein are hairpin adapters that can be used to close via ligation, for example, a double-stranded amplicon thereby forming a double-stranded DNA target nucleic acid molecule. A hairpin adapter can be a fragment of DNA of any length having an overhang portion to close double-stranded circular DNA.

Hairpin adapters can be nucleic acids. Hairpin adapters can be DNA or RNA. The nucleic acid can include one or two strands of any synthetic nucleic acid known in the art, including peptide nucleic acid (PNA), glycerol nucleic acid (GNA), threose nucleic acid (TNA), locked nucleic acid (LNA) or other synthetic polymers with nucleotide side chains. The hairpin adapters comprise a single-stranded portion (e.g., loop region) and double-stranded portion. The single-stranded portion can be in a loop formation. The double-stranded portion can be linear and having a portion that can be complementary to the terminal overhang of the double-stranded amplicon. In other words, the linear portion of the hairpin adapter can be partially double-stranded and can include an overhang portion complementary to the overhang portion in the double-stranded amplicon. In an aspect, the first hairpin adapter comprises a first sequence complementary to the first terminal overhang of the double-stranded amplicon at the 3′ end of the double-stranded amplicon. In an aspect, the second hairpin adapter comprises a second sequence complementary to the second terminal overhang at the 3′ end of the double-stranded amplicon.

Generally, the first hairpin adapter can be attached to one terminus of the amplicon and the second hairpin adapter can be attached to a second and different terminus of the amplicon.

The region of double-stranded nucleic acid can be any length. The region can typically be about 40 or fewer base pairs, such as 30 or fewer base pairs, 20 or fewer base pairs or 10 or fewer base pairs, in length. The region is preferably 5 to 20 base pairs in length and more preferably 6 to 10 base pairs in length. In an aspect, the region of double-stranded nucleic acid is 6 base pairs in length. For example, the underlined regions of the two constructs below (SEQ ID NO: 6, top; SEQ ID NO: 7, bottom) show adaptors that when combined would have one or more double-stranded nucleic acid regions of 6 base pairs.

Pho5′ CCGTCGGTCCCAAACTACACCCCAACCTGCATACCTCGACGGTA
GCAACT 50 nt
Pho 5′ GCGAGCCCCACCCACCCACCCACCCACCCACCCAGCTCGCACT
TCAAT48 nt

This region of double-stranded nucleic acid can be formed by hybridization of two separate strands of single-stranded nucleic acid. The two separate strands can be the same type of nucleic acid or different types of nucleic acid as long as they hybridize. The two separate strands can be any of the types of nucleic acid described herein. Suitable conditions that allow hybridization of nucleic acids are known to those of ordinary skill in the art.

The region of double-stranded nucleic acid can be formed by hybridization of two separated regions of a single-stranded nucleic acid such that the adapter comprises a hairpin loop. The formation of hairpin loops is known in the art. For example, the hairpin adapter loop portion can be formed from a single-stranded nucleic acid. The hairpin adapter loop portion can be the same type of nucleic acid as that making up the region of the double-stranded nucleic acid. Alternatively, the hairpin adapter loop portion can be a different type of nucleic acid from that making up the region of double-stranded nucleic acid. The hairpin adapter loop portion can be any of the types of nucleic acid described herein.

The nucleic acid in the adapter can be chosen such that the adapters can be capable of ligating to the double-stranded nucleic acid that can result after a series of steps (see below) to form a target nucleic acid molecule for sequencing.

The hairpin adapter loop portion can be any length. In an aspect, the hairpin adapter loop portion can be 50 or fewer bases, such as 40 or fewer bases, 30 or fewer bases, 20 or fewer bases or 10 or fewer bases, in length. In an aspect, the hairpin adapter loop portion can be from about 1 to 50, from 2 to 40 or from 6 to 30 bases in length. In an aspect, longer lengths of the hairpin adapter loop portion can be from 15 to 50 bases. Similarly, shorter lengths of the hairpin adapter loop portion can be from 1 to 5 bases.

In one aspect, the hairpin adapters can each have a length in the range of from 8 to 60 nucleotides; in another aspect, they have a length in the range of from 8 to 32 nucleotides; in another aspect, they have a length in a range selected from about 4 to about 400 nucleotides; from about 10 to about 100 nucleotides, from about 400 to about 4000 nucleotides, from about 10 to about 80 nucleotides, from about 20 to about 70 nucleotides, from about 30 to about 60 nucleotides, and from about 4 to about 10 nucleotides. In an aspect, the hairpin adapters can have a total length from about 20 to about 30 bases.

The double-stranded portion of the hairpin adapter can have one free end as the other end can be closed by the hairpin adapter loop portion. The free end can ligate to a double-stranded nucleic acid amplicon.

The free end of the double-stranded portion of the hairpin adapter can be in any form. In an aspect, the free end of the double-stranded portion of the hairpin adapter can be sticky. As described herein, the free ends of the double-stranded portion of the hairpin adapters need to be sticky and complementary to one particular adapter in order to direct the adapter to the correct portion of the double stranded DNA fragment. If blunt end are used, the same adapter can be used to ligate at both ends of the hairpin adapter and therefore the two sequencing primer regions located in the loop portion can be the same from both directions or both strands which is not desired. In some aspects, the hairpin can be designed to contain an overhanging portion and therefore negate the need for a restriction enzyme binding site. In other words, the free end of the double-stranded portion of the hairpin adapter does not have to form a base pair. In an aspect, the sticky end may have a 5′ or 3′ overhang. In an aspect, the hairpin adapters comprise restriction enzyme binding sites. In an aspect, the first hairpin adapter comprises a first restriction enzyme site. In an aspect, the second hairpin adapter comprises a second restriction enzyme site. In an aspect, the restriction enzyme binding site is a Type II restriction endonuclease site. In some aspects, the Type II endonuclease restriction site of the first hairpin adapter is not the same as the Type II endonuclease restriction site of the second hairpin adaptor. In an aspect, the hairpin adapter comprises a restriction endonuclease site, which cuts outside of the recognition sequence. The overhang portion should be sticky and should be different for each adapter to assure directionality of ligation. In an aspect, an enzyme can used to ligate the two ends of the linear strand comprising the adapter and the amplicon to form a partially circularized nucleic acid. This can be done using a single step. In an aspect, a second adapter can be added to the other terminus of the amplicon followed by ligation. In an aspect, a second circular sequence can be formed resulting in a dumbbell configuration having a double-stranded portion and two single-stranded portions.

In an aspect, the hairpin adapters comprise a primer binding site. In an aspect, the first hairpin adapter comprises a first primer binding site. In an aspect, the second hairpin adapter comprises a second primer binding site. In an aspect, the first hairpin adapter and the second hairpin adapter are different.

In general, the primer binding sequence can be from about 3 to about 40 nucleotides in length. In an aspect, the primer binding sequence can be from about 15 to about 25 nucleotides in length. Primer oligonucleotides are usually 6 to 25 bases length. In an aspect, the primer binding sequence can be contained within any of the other hairpin adapter sequences.

The nucleic acids or regions that are capable of hybridizing to one another can be at least 80%, at least 85%, at least 90%, at least 95%, at least 98%, at least 99% homology based on sequence identity. In an aspect, the nucleic acids or regions can be complementary (e.g. share 100% homology based on sequence identity). Standard methods in the art may be used to determine homology.

In an aspect, the first hairpin adapter comprises a first sequence complementary to the first terminal overhang of the double-stranded amplicon. In an aspect, the second hairpin adapter comprises a second sequence complementary to the second terminal overhang at the 3′ end of the double-stranded amplicon. The double-stranded amplicon comprises two strands of DNA, a sense and an anti-sense strand. Each strand can also have a 5′ and 3′ end or region. “Terminal overhang” refers to a portion of the double-stranded amplicon. In some aspects, the terminal overhang can be single-stranded. In an aspect, the terminal overhang can be the 3′ end of the double-stranded amplicon. In an aspect, a first terminal overhang and a second terminal overhang can each be one of the 3′ ends of the sense or anti-sense strands of the double-stranded amplicon. Unless specified, the terms “first” and “second” are not meant to confer a specific strand of the double-stranded amplicon, such that the first terminal overhang can be the sense strand or the anti-sense strand. The use of hairpin adapters in the double-stranded amplicons can be important to form a closed double-stranded circular DNA which becomes the template for rolling circle amplification. In an aspect, this closed double-stranded circular DNA can be in a dumbbell configuration serving as a template comprising the sequence of interest.

Hairpin adapters can vary widely in length, and can depend in part on the number and type of functional elements desired. Examples of functional elements include, but are not limited to, anchor sequences, sequences complementary to capture probe sequences (e.g. for attachment to surfaces), tagging sequences, secondary structure sequences, sequences for attachment/hybridization of label probes, functionalization sequences, primer binding sites, recognition sites for nucleases, such as nicking enzymes, restriction endonucleases, and the like. In an aspect, the hairpin adapters comprise a restriction endonuclease recognition site as known in the art. In one embodiment, such recognition sites can be for nicking enzymes.

In an aspect, the restriction endonuclease site can be a Type IIs restriction endonuclease site. Type IIs endonucleases are generally commercially available and are well-known in the art. Type IIs endonucleases recognize specific sequences of nucleotide base pairs within a double-stranded polynucleotide sequence. Upon recognizing that sequence, the endonuclease can cleave the polynucleotide sequence, generally leaving an overhang of one strand of the sequence, or “sticky end.” Type IIs endonucleases also generally cleave outside of their recognition sites; the distance can be anywhere from 2 to 20 nucleotides away from the recognition site. Because the cleavage occurs within an ambiguous portion of the polynucleotide sequence, it can permit the capturing of the ambiguous sequence up to the cleavage site. Usually, type IIs restriction endonucleases can be selected that have cleavage sites separated from their recognition sites by at least six nucleotides (i.e., the number of nucleotides between the end of the recognition site and the closest cleavage point). Examples of type IIs restriction endonucleases include, but are not limited to, Eco57M I, Mme I, Acu I, Bpm I, BceA I, Bbv I, BciVI, BpuE I, BseM II, BseR I, Bsg I, BsmF I, BtgZ I, Eci I, EcoP15 I, Eco57M I, Fok I, Hga I, Hph I, Mho II, Mnl I, SfaN I, TspDT I, TspDW I, Taq II, and the like.

In an aspect, each hairpin adapter comprises the same Type IIs restriction endonuclease site. Alternatively, different hairpin adapters comprise different sites.

### D. Amplicon

An amplicon can be a fragment of DNA or RNA or nucleic acid comprising the sequence of interest. In an aspect, the amplicon can be double-stranded. In an aspect, the amplicon comprises the sequence of interest. The amplicon can comprise a first and a second strand. In some aspects, the amplicon can be amplified and contacted with primers comprising uracil residues, for instance, at the 3′ end, to insert the uracil residues for later cleavage, generating a modified amplicon.

In an aspect, the amplicon can comprise a terminal overhang region. In an aspect, the double-stranded amplicon comprises two terminal overhang regions located at the 3′ end of each of the strands. These cohesive ends can be ligated, in a specific orientation to two hairpin adapters (discussed above and below). In an aspect, the amplicon can be transformed after ligation with the two hairpin adapters to form a template that can be in the dumbbell circle formation for rolling circle amplification.

In an aspect, a plurality of amplicons can be immobilized on a surface. In an aspect, amplicons are generated for disposal onto an array.

In an aspect, the amplicons generated herein can comprise two or more concatemers, wherein the concatemers comprise a first hairpin adapter, at least one sequence of interest and a second hairpin adapter. In an aspect, the density of the amplicons on a surface can be at least 100 per square millimeter, at least 10,000 per square millimeter or at least 100,000 amplicons per square millimeter. In an aspect, the density of the amplicons on a surface can be more than 100,000, 200,000, 300,000, 400,000, 500,000, 600,000 amplicons per square millimeter or any number in between.

### E. Primers and Primer Sets

Any sequence can serve as a primer binding sequence, to bind a primer, as any double-stranded sequence can be recognized by the polymerase. In general, the primer binding sequence can be from about 3 to about 30 nucleotides in length, about 15 to about 25 in length. Primer oligonucleotides can be usually 6 to 25 bases in length. The primer binding sequence can be contained within any of the other hairpin adapter sequences.

The sequence in a primer can hybridize to another nucleic acid molecule and can be referred to as the complementary portion of the primer. The complementary portion of a primer can be any length that supports specific and stable hybridization between the primer and the nucleic acid molecules under the reaction conditions.

The primers can have, for example, a length of 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, or 40 nucleotides.

The primers can have, for example, a length of less than 4 nucleotides, less than 5 nucleotides, less than 6 nucleotides, less than 7 nucleotides, less than 8 nucleotides, less than 9 nucleotides, less than 10 nucleotides, less than 11 nucleotides, less than 12 nucleotides, less than 13 nucleotides, less than 14 nucleotides, less than 15 nucleotides, less than 16 nucleotides, less than 17 nucleotides, less than 18 nucleotides, less than 19 nucleotides, less than 20 nucleotides, less than 21 nucleotides, less than 22 nucleotides, less than 23 nucleotides, less than 24 nucleotides, less than 25 nucleotides, less than 26 nucleotides, less than 27 nucleotides, less than 28 nucleotides, less than 29 nucleotides, less than 30 nucleotides, less than 31 nucleotides, less than 32 nucleotides, less than 33 nucleotides, less than 34 nucleotides, less than 35 nucleotides, less than 36 nucleotides, less than 37 nucleotides, less than 38 nucleotides, less than 39 nucleotides, or less than 40 nucleotides.

### F. Rolling Circle Amplification Primer

The methods disclosed herein can include rolling circle amplification. In RCA, amplification occurs with each rolling circle amplification primer, thereby forming a concatemer of tandem repeats (i.e., a TS-DNA) of segments complementary to the first-stage amplification target circle (ATC) being replicated by each primer. Bipolar primers can be used as second-stage primers. Since the bipolar primers have a 3′-OH at each end, they are automatically in the proper orientation for use as a primer for additional stages of amplification. In addition, because the bipolar primers have a 3′-OH at each end, they serve to curtail any strand displacement that might otherwise occur. Further, because of the presence of a 3′-OH at each end of the bipolar primer, the TS-DNA and second-stage, or higher order, ATCs (second-stage ATC, third-stage ATC, forth-stage ATC, and so on) complementary sequences can be arranged in any configuration within the primer sequence.

### G. DNA Strand Displacement Primers

Primers used for secondary DNA strand displacement are referred to herein as DNA strand displacement primers. One form of DNA strand displacement primer, referred to herein as a secondary DNA strand displacement primer, is an oligonucleotide having sequence matching part of the sequence of an ATC. This sequence is referred to as the matching portion of the secondary DNA strand displacement primer. This matching portion of a secondary DNA strand displacement primer is complementary to sequences in TS-DNA. The matching portion of a secondary DNA strand displacement primer may be complementary to any sequence in TS-DNA. The matching portion of a secondary DNA strand displacement primer can be any length that supports specific and stable hybridization between the primer and its complement. Generally this is 12 to 35 nucleotides long, but is can be 18 to 25 nucleotides long.

Another form of DNA strand displacement primer, referred to herein as a tertiary DNA strand displacement primer, is an oligonucleotide having sequence complementary to part of the sequence of an ATC. This sequence is referred to as the complementary portion of the tertiary DNA strand displacement primer. This complementary portion of the tertiary DNA strand displacement primer matches sequences in TS-DNA. The complementary portion of a tertiary DNA strand displacement primer can be complementary to any sequence in the ATC. The complementary portion of a tertiary DNA strand displacement primer can be any length that supports specific and stable hybridization between the primer and its complement. Generally, this is 12 to 35 nucleotides long, but is preferably 18 to 25 nucleotides long.

DNA strand displacement primers and their use are described in more detail in U.S. Pat. No. 5,854,033 and WO 97/19193.

H. DNA polymerases

DNA polymerases useful in the rolling circle replication step of RCA must perform rolling circle replication of primed single-stranded circles. Such polymerases are referred to herein as rolling circle DNA polymerases. For rolling circle replication, a DNA polymerase can displace the strand complementary to the template strand, termed strand displacement, and lacks a 5′ to 3′ exonuclease activity. Strand displacement results in synthesis of multiple tandem copies of an amplification target circle. A 5′ to 3′ exonuclease activity, if present, might result in the destruction of the synthesized strand. In an aspect, DNA polymerases for use in the disclosed method can be processive. The suitability of a DNA polymerase for use in the disclosed method can be readily determined by assessing its ability to carry out rolling circle replication. In an aspect, the rolling circle DNA polymerases can be bacteriophage ϕ29 DNA polymerase (U.S. Pat. Nos. 5,198,543 and 5,001,050 to Blanco et al.), phage M2 DNA polymerase (Matsumoto et al., Gene 84:247 (1989)), phage ϕPRD1 DNA polymerase (Jung et al., Proc. Natl. Acad. Sci. USA 84:8287 (1987)), VENT® DNA polymerase (Kong et al., J. Biol. Chem. 268:1965-1975 (1993)), Klenow fragment of DNA polymerase I (Jacobsen et al., Eur. J. Biochem. 45:623-627 (1974)), T5 DNA polymerase (Chatterjee et al., Gene 97:13-19 (1991)), PRD1 DNA polymerase (Zhu and Ito, Biochim. Biophys. Acta. 1219:267-276 (1994)), modified T7 DNA polymerase (Tabor and Richardson, J. Biol. Chem. 262:15330-15333 (1987); Tabor and Richardson, J. Biol. Chem. 264:6447-6458 (1989); Sequenase™ (U.S. Biochemicals)), T7 native polymerase, Bst polymerase, and T4 DNA polymerase holoenzyme (Kaboord and Benkovic, Curr. Biol. 5:149-157 (1995)).

Strand displacement can be facilitated through the use of a strand displacement factor, such as helicase. It is considered that any DNA polymerase that can perform rolling circle replication in the presence of a strand displacement factor is suitable for use in the disclosed method, even if the DNA polymerase does not perform rolling circle replication in the absence of such a factor. Strand displacement factors useful in RCA include BMRF1 polymerase accessory subunit (Tsurumi et al., J. Virology 67(12):7648-7653 (1993)), adenovirus DNA-binding protein (Zijderveld and van der Vliet, J. Virology 68(2):1158-1164 (1994)), herpes simplex viral protein ICP8 (Boehmer and Lehman, J. Virology 67(2):711-715 (1993); Skaliter and Lehman, Proc. Natl. Acad. Sci. USA 91(22):10665-10669 (1994)), single-stranded DNA binding proteins (SSB; Rigler and Romano, J. Biol. Chem. 270:8910-8919 (1995)), and calf thymus helicase (Siegel et al., J. Biol. Chem. 267:13629-13635 (1992)).

The ability of a polymerase to carry out rolling circle replication can be determined by using the polymerase in a rolling circle replication assay such as those described in Fire and Xu, Proc. Natl. Acad. Sci. USA 92:4641-4645 (1995), and in Lizardi (U.S. Pat. No. 5,854,033, e.g., Example 1 therein).

Additional examples of commercially available polymerases that can be used in the methods that utilize the concatamers disclosed herein include, but are not limited to, Therminator I-III. These polymerases are derived from Thermococcus sp. and carry mutations allowing for incorporation of modified nucleotides. Such polymerases can be sued in the sequencing methods disclosed herein.

I. dNTPs

In any of the embodiments of the instant disclosure, dNTPs can be members selected from the group consisting of dUTP, dCTP, dATP, dGTP, a naturally occurring dNTP different from the foregoing, an analog of a dNTP, and a dNTP having a universal base.

The materials described above can be packaged together in any suitable combination as a kit useful for performing the disclosed method. For example, a kit can include a plurality of reporter binding primers and/or a plurality of analyte capture agents. The analyte capture agents in the kit can be associated with a solid support.

### J. Analyte Capture Agents

The disclosed methods can be performed using any analyte. Analytes can be nucleic acids, including but not limited to amplified nucleic acids such as TS-DNA. An analyte capture agent (or probe) is any compound that can interact with an analyte and allow the analyte to be immobilized or separated from other compounds and analytes. An analyte capture agent includes an analyte interaction portion. Analyte capture agents can also include a capture portion. Analyte capture agents without a capture portion preferably are immobilized on a solid support. The analyte interaction portion of an analyte capture agent is a molecule that interacts specifically with a particular molecule or moiety. The molecule or moiety that interacts specifically with an analyte interaction portion can be an analyte or another molecule that serves as an intermediate in the interaction between the analyte interaction portion and the analyte. It is to be understood that the term analyte refers to both separate molecules and to portions of molecules, such as an epitope of a protein that interacts specifically with an analyte interaction portion. Antibodies, either member of a receptor/ligand pair, and other molecules with specific binding affinities are examples of molecules that can be used as an analyte interaction portion of an analyte capture agent. The specific binding portion of an analyte capture agent can also be any compound or composition with which an analyte can interact, such as peptides. An analyte capture agent that interacts specifically with a particular analyte is said to be specific for that analyte. For example, an analyte capture agent with an analyte interaction portion that is an antibody that binds to a particular antigen is said to be specific for that antigen. The antigen is the analyte.

Examples of molecules useful as the analyte interaction portion of analyte capture agents are antibodies, such as crude (serum) antibodies, purified antibodies, monoclonal antibodies, polyclonal antibodies, synthetic antibodies, antibody fragments (for example, Fab fragments); antibody interacting agents, such as protein A, carbohydrate binding proteins, and other interactants; protein interactants (for example avidin and its derivatives); peptides; and small chemical entities, such as enzyme substrates, cofactors, metal ions/chelates, and haptens. Antibodies may be modified or chemically treated to optimize binding to surfaces and/or targets.

Antibodies useful as the analyte interaction portion of analyte capture agents can be obtained commercially or produced using well-established methods. For example, Johnstone and Thorpe, on pages 30-85, describe general methods useful for producing both polyclonal and monoclonal antibodies. The entire book describes many general techniques and principles for the use of antibodies in assay systems.

The capture portion of an analyte capture agent is any compound that can be associated with another compound. Preferably, a capture portion is a compound, such as a ligand or hapten that binds to or interacts with another compound, such as ligand-binding molecule or an antibody. It is also preferred that such interaction between the capture portion and the capturing component be a specific interaction, such as between a hapten and an antibody or a ligand and a ligand-binding molecule. Examples of haptens include biotin, FITC, digoxigenin, and dinitrophenol. The capture portion can be used to separate compounds or complexes associated with the analyte capture agent from those that do not.

Capturing analytes or analyte capture agents on a substrate can be accomplished in several ways. In one embodiment, capture docks are adhered or coupled to the substrate. Capture docks are compounds or moieties that mediate adherence of an analyte by binding to, or interacting with, the capture portion on an analyte capture agent (with which the analyte is, or will be, associated). Capture docks immobilized on a substrate allow capture of the analyte on the substrate. Such capture provides a convenient means of washing away reaction components that might interfere with subsequent steps. Alternatively, analyte capture agents can be directly immobilized on a substrate. In this case, the analyte capture agent need not have a capture portion.

In one embodiment, the analyte capture agent or capture dock to be immobilized can be an anti-hybrid antibody. Methods for immobilizing antibodies and other proteins to substrates are well established. Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. A preferred attachment agent is a heterobifunctional cross-linking agent such as N-[γ-maleimidobutyryloxy]succinimide ester (GMBS). These and other attachment agents, as well as methods for their use in attachment, are described in Protein immobilization: fundamentals and applications, Richard F. Taylor, ed. (M. Dekker, New York, 1991), Johnstone and Thorpe, Immunochemistry In Practice (Blackwell Scientific Publications, Oxford, England, 1987) pages 209-216 and 241-242, and Immobilized Affinity Ligands, Craig T. Hermanson et al., eds. (Academic Press, New York, 1992). Antibodies can be attached to a substrate by chemically cross-linking a free amino group on the antibody to reactive side groups present within the substrate. For example, antibodies may be chemically cross-linked to a substrate that contains free amino, carboxyl, or sulfur groups using glutaraldehyde, carbodiimides, or heterobifunctional agents such as GMBS as cross-linkers. In this method, aqueous solutions containing free antibodies are incubated with the solid-state substrate in the presence of glutaraldehyde or carbodiimide. For crosslinking with glutaraldehyde the reactants can be incubated with 2% glutaraldehyde by volume in a buffered solution such as 0.1 M sodium cacodylate at pH 7.4. Other standard immobilization chemistries are known by those of skill in the art.

One useful form of analyte capture agents are peptides. When various peptides are immobilized in an array, they can be used as “bait” for analytes. For example, an array of different peptides can be used to access whether a sample has analytes that interact with any of the peptides. Comparisons of different samples can be made by, for example, noting differences in the peptides to which analytes in the different samples become associated. In another form of the disclosed method, an array of analyte capture agents specific for analytes of interest can be used to access the presence of a whole suite of analytes in a sample.

In use, the analyte capture agents need not be absolutely pure. The analyte capture agents preferably are at least 20% pure, more preferably at least 50% pure, more preferably at least 80% pure, and more preferably at least 90% pure.

One or more analyte capture agents can be used with the methods described herein. For example, one or more first analyte capture agents and one or more second analyte capture agents can be mixed. Mixing of one or more first analyte capture agents and the one or more second analyte capture agents can be accomplished by associating, simultaneously or sequentially, the one or more first analyte capture agents and the one or more second analyte capture agents with the same solid support.

### K. Detection Labels

To aid in detection and quantitation of nucleic acids amplified using the disclosed methods, detection labels can be directly incorporated into amplified nucleic acids or can be coupled to detection molecules. As used herein, a detection label is any molecule that can be associated with amplified nucleic acid, directly or indirectly, and which results in a measurable, detectable signal, either directly or indirectly. Many such labels for incorporation into nucleic acids or coupling to nucleic acid or antibody probes are known to those of skill in the art. Examples of detection labels suitable for use in RCA are radioactive isotopes, fluorescent molecules, phosphorescent molecules, enzymes, antibodies, and ligands.

Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, Oreg. and Research Organics, Cleveland, Ohio.

Labeled nucleotides are preferred form of detection label since they can be directly incorporated into the products of RCA during synthesis. Examples of detection labels that can be incorporated into amplified DNA or RNA include nucleotide analogs such as BrdUrd (Hoy and Schimke, Mutation Research 290:217-230 (1993)), BrUTP (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (BUDR triphosphate, Sigma), and a preferred nucleotide analog detection label for RNA is Biotin-16-uridine-5′-triphosphate (Biotin-16-dUTP, Boehringher Mannheim). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into amplified nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo[3.3.1.1^{3,7}]decane]-4-yl) phenyl phosphate; CDP-Star® (disodium 2-chloro-5-(4-methoxyspiro {1,2-dioxetane-3-2′-(5′-chloro)tricyclo[3.3.1.1^{3,7}]decan}-4-yl) phenyl phosphate) and AMPPD® (disodium 3-(4-methoxyspiro {1,2-dioxetane-3-2′-tricyclo[3.3.1.1^{3,7}]phenyl phosphate) (all available from Tropix, Inc.).

A preferred detection label for use in detection of amplified RNA is acridinium-ester-labeled DNA probe (GenProbe, Inc., as described by Arnold et al., Clinical Chemistry 35:1588-1594 (1989)). An acridinium-ester-labeled detection probe permits the detection of amplified RNA without washing because unhybridized probe can be destroyed with alkali (Arnold et al. (1989)).

Molecules that combine two or more of these detection labels are also considered detection labels. Any of the known detection labels can be used with the disclosed probes, tags, and method to label and detect nucleic acid amplified using the disclosed method. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a scanner or spectrophotometer, or directly visualized with a camera; enzymes can be detected by detection or visualization of the product of a reaction catalyzed by the enzyme; antibodies can be detected by detecting a secondary detection label coupled to the antibody. Such methods can be used directly in the disclosed method of amplification and detection. As used herein, detection molecules are molecules that interact with amplified nucleic acid and to which one or more detection labels are coupled.

Other examples of molecules for use in detecting any of the TS-DNA products formed according to the methods described herein, include, but are not limited to, decorators, or decorating agents, including hybridization probes, any of the fluorescent agents disclosed herein, ligand binding molecules (such as avidin), antibodies, FKBP fold binding molecules (such as rapamycin), enzymes, receptors, nucleic acid binding proteins (such as transcription factors), ribosomal or other RNA binding proteins, affinity agents (such as aptamers, which are nucleic acids with affinity for small molecule ligands [See: Marshall et al, Current Biology, 5, 729-734 (1997) for a review], and other agents known to those skilled in the art and suitable for conjugation with an CA primer or detection tag.

### L. Detection Probes

Detection probes can be used in the methods disclosed herein. Detection probes are labeled oligonucleotides having sequence complementary to detection tags on TS-DNA. The complementary portion of a detection probe can be any length that supports specific and stable hybridization between the detection probe and the detection tag. For this purpose, a length of 10 to 35 nucleotides is preferred, with a complementary portion of a detection probe 16 to 20 nucleotides long being most preferred. Detection probes can contain any of the detection labels described above. Preferred labels are biotin and fluorescent molecules. In an aspect, the detection probe can be a molecular beacon. Molecular beacons are detection probes labeled with fluorescent moieties where the fluorescent moieties fluoresce only when the detection probe is hybridized (Tyagi and Kramer, Nature Biotechnology 14:303-308 (1996)). The use of such probes eliminates the need for removal of unhybridized probes prior to label detection because the unhybridized detection probes will not produce a signal. This can be useful in multiplex assays. The TS-DNA can be collapsed as described in WO 97/19193 using collapsing detection probes. Collapsing TS-DNA can be useful with combinatorial multicolor coding.

### M. Structure of Probes

As used herein, a “probe” can mean an oligonucleotide used in hybridization or in litigation of two probes, a detection probe or capture probe. In an aspect, probes can be labeled oligonucleotides having sequence complementary to detection tags or another sequence on amplified nucleic acids. The complementary portion of a probe can be any length that supports specific and stable hybridization between the probe and its complementary sequence on the amplified DNA.

In an aspect, the length of the probe can vary. In an aspect, the probe can have a few specific bases and many degenerate bases. In an aspect, the length of the probe can be between 10 to 35 nucleotides, with a complementary portion of the probe being about 16 to 20 nucleotides long.

The probes as described herein can be labeled in a variety of ways including but not limited to direct or indirect attachment of radioactive moieties, fluorescent moieties, calorimetric moieties, and chemiluminescent moieties. Probes can contain any of the detection labels described herein. Examples of detection labels include but are not limited to biotin, fluorescent molecules, and a molecular beacon. Molecular beacons are probes labeled with fluorescent moieties where the fluorescent moieties fluoresce only when the detection probe is hybridized (Tyagi and Kramer, Nature Biotechnol. 14:303-309 (1995)). The use of such probes eliminates the need for removal of unhybridized probes prior to label detection because the unhybridized detection probes will not produce a signal.

In an aspect, probes can be coupled, directly or via a spacer molecule, to a solid support.

### N. Support

A wide variety of supports can be used for arrays. In an aspect, supports can be rigid solids that have a surface, such as, for example, a substantially planar surface so that single molecules to be interrogated can be in the same plane. The latter feature permits efficient signal collection by detection optics.

In an aspect, solid supports can be nonporous, particularly when random arrays of single molecules are analyzed by hybridization reactions requiring small volumes. Suitable solid support materials include materials such as glass, polyacrylamide-coated glass, ceramics, silica, silicon, quartz, various plastics, and the like.

In an aspect, the area of a planar surface can be in the range of from 0.5 to 4 cm². In an aspect, the solid support can be glass or quartz, such as a microscope slide, having a surface that can be uniformly silanized. This can be accomplished using conventional protocols, e.g. acid treatment followed by immersion in a solution of 3-glycidoxypropyl trimethoxysilane, N, N-diisopropylethylamine, and anhydrous xylene (8:1:24 v/v) at 80° C., which forms an epoxysilanized surface. Such a surface can be readily treated to permit end-attachment of capture oligonucleotides, e.g. by providing capture oligonucleotides with a 3′ or 5′ triethylene glycol phosphoryl spacer prior to application to the surface. Many other protocols can be used for adding reactive functionalities to glass and other surfaces.

### O. Solid Supports

Solid supports are solid-state substrates or supports with which analytes or concatamers can be associated. Analytes can be associated with solid supports directly or indirectly. For example, analytes can be directly immobilized on solid supports. Analyte capture agents and accessory molecules can also be immobilized on solid supports. In an aspect, the solid support can be an array. Another form of solid support is an array detector. An array detector is a solid support to which multiple different address probes or detection molecules have been coupled in an array, grid, or other organized pattern.

Solid-state substrates for use in solid supports can include any solid material to which oligonucleotides can be coupled. This includes materials such as acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates can also comprise at least two thin films, membranes, bottles, dishes, fibers, woven fibers, shaped polymers, particles, beads, microparticles, or a combination thereof. Solid-state substrates and solid supports can be porous or non-porous. Additional arrangements are described in U.S. Pat. No. 5,854,033, which is hereby incorporated by reference for its teaching of said additional arrangements. In an aspect, the solid-state substrate is a microtiter dish. In an aspect, the microtiter dish can be the standard 96-well type. In other aspects, a multiwell glass slide can be employed that normally contains one array per well. This feature allows for greater control of assay reproducibility, increased throughput and sample handling, and ease of automation.

Different analytes, analyte capture agents, or accessory molecules can be used together as a set. The set can be used as a mixture of all or subsets of the analytes, analyte capture agents, and accessory molecules used separately in separate reactions, or immobilized in an array. Analytes, analyte capture agents, and accessory molecules used separately or as mixtures can be physically separable through, for example, association with or immobilization on a solid support. An array includes a plurality of analytes, analyte capture agents and/or accessory molecules immobilized at identified or predefined locations on the array. Each of the different predefined regions of a solid support can be physically separated from each other. Each predefined location on the array generally has one type of component (that is, all the components at that location are the same). Each location can have multiple copies of the component. The spatial separation of different components in the array allows separate detection and identification of analytes.

Although preferred, it is not required that a given array be a single unit or structure. The set of analytes, analyte capture agents, or accessory molecules can be distributed over any number of solid supports. For example, at one extreme, each probe can be immobilized in a separate reaction tube or container, or on separate beads or microparticles.

Different modes of the disclosed method can be performed with different components (for example, analytes, analyte capture agents, and accessory molecules) immobilized on a solid support.

In alternative embodiments, RCA can be performed in solution, and the products of the amplification can be captured on an array. For example, a biotinylated capture antibody can be added to a sample containing the analyte, followed by a reporter binding primer that can bind to a different location on the analyte. These components—the capture antibody and the reporter binding primer—can be added in any order. RCA can then be performed to produce TS-DNA, and purified on a matrix containing streptavidin (streptavidin beads (Dynal), for example). The TS-DNA can then be detected or quantitated by hybridization to an array containing oligonucleotide probes complementary to the TS-DNA. Such probes are referred to herein as address probes. By attaching different address probes to different regions of a solid support, different RCA products can be captured at different, and therefore diagnostic, locations on the solid support. For example, in a microtiter plate multiplex assay, address probes specific for up to 96 different TS-DNAs (each amplified via different primers and ATCs) can be immobilized on a microtiter plate, each in a different well. Capture and detection can occur in those probe elements in the array corresponding to TS-DNAs for which the corresponding analytes were present in a sample.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), Khrapko et al., Mol Biol (Mosk) (USSR) 25:718-730 (1991), and Guo et al., Nucleic Acids Res. 22:5456-5465 (1994). A method for immobilization of 3′-amine oligonucleotides on casein-coated slides is described by Stimpson et al., Proc. Natl. Acad. Sci. USA 92:6379-6383 (1995). A preferred method of attaching oligonucleotides to solid-state substrates is described by Guo et al., Nucleic Acids Res. 22:5456-5465 (1994).

Some solid supports useful in RCA assays and the methods disclosed herein have detection antibodies attached to a solid-state substrate. Such antibodies can be specific for a molecule of interest. Captured molecules of interest can then be detected by binding of a second, reporter antibody, followed by RCA. Such a use of antibodies in a solid support allows RCA assays to be developed for the detection of any molecule for which antibodies can be generated. Methods for immobilizing antibodies to solid-state substrates are well established. Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. In an aspect, the attachment agent can be the heterobifunctional cross-linker N-[γ-Maleimidobutyryloxy] succinimide ester (GMBS). These and other attachment agents, as well as methods for their use in attachment, are described in Protein immobilization: fundamentals and applications, Richard F. Taylor, ed. (M. Dekker, New York, 1991), Johnstone and Thorpe, Immunochemistry In Practice (Blackwell Scientific Publications, Oxford, England, 1987) pages 209-216 and 241-242, and Immobilized Affinity Ligands, Craig T. Hermanson et al., eds. (Academic Press, New York, 1992). Antibodies can be attached to a substrate by chemically cross-linking a free amino group on the antibody to reactive side groups present within the solid-state substrate. For example, antibodies may be chemically cross-linked to a substrate that contains free amino, carboxyl, or sulfur groups using glutaraldehyde, carbodiimides, or GMBS, respectively, as cross-linker agents. In this method, aqueous solutions containing free antibodies are incubated with the solid-state substrate in the presence of glutaraldehyde or carbodiimide.

A method for attaching antibodies or other proteins to a solid-state substrate is to functionalize the substrate with an amino- or thiol-silane, and then to activate the functionalized substrate with a homobifunctional cross-linker agent such as (Bis-sulfo-succinimidyl suberate (BS³) or a heterobifunctional cross-linker agent such as GMBS. For cross-linking with GMBS, glass substrates are chemically functionalized by immersing in a solution of mercaptopropyltrimethoxysilane (1% vol/vol in 95% ethanol pH 5.5) for 1 hour, rinsing in 95% ethanol and heating at 120° C. for 4 hrs. Thiol-derivatized slides are activated by immersing in a 0.5 mg/ml solution of GMBS in 1% dimethylformamide, 99% ethanol for 1 hour at room temperature. Antibodies or proteins are added directly to the activated substrate, which are then blocked with solutions containing agents such as 2% bovine serum albumin, and air-dried. Other standard immobilization chemistries are known by those of skill in the art.

Each of the components (analyte capture agents, accessory molecules, and/or analytes) immobilized on the solid support can be located in a different predefined region of the solid support. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. In an aspect, components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

### P. Structure of Random Arrays

In an aspect, analytes or concatemers can be fixed to a surface by any of a variety of techniques, including covalent attachment and non-covalent attachment. In an aspect, the surface can have attached sequencing probes or capture oligonucleotides that form complexes, e.g. double-stranded duplexes, with a segment of a hairpin adapter oligonucleotide in the concatemers, such as an anchor binding site or other elements disclosed herein. In other aspects, capture oligonucleotides can comprise oligonucleotide clamps, or like structures, that form triplexes with adapter oligonucleotides. In another aspect, the surface can have reactive functionalities that react with complementary functionalities on the concatemers to form a covalent linkage, e.g. by way of the same techniques used to attach cDNAs to microarrays. Long DNA molecules, e.g. several hundred nucleotides or larger, can also be efficiently attached to hydrophobic surfaces, such as a clean glass surface that has a low concentration of various reactive functionalities, such as —OH groups.

### Methods

Disclosed herein are compositions and methods for detecting nucleotide sequence information from a sequence of interest or target sequences using hairpin adapters to form a partially double-stranded circular DNA.

A method of enzymatically synthesizing a polynucleotide of a predetermined sequence in a stepwise manner using reversibly 3′0H-blocked nucleoside triphosphates was described by Hiatt and Rose (U.S. Pat. No. 5,990,300). They disclose besides esters, ethers, carbonitriles, phosphates, phosphoramides, carbonates, carbamates, borates, sugars, phosphoramidates, phenylsulfenates, sulfates and sulfones also nitrates as cleavable 3′0H-protecting group. The deprotection may be carried out by chemical or enzymatic means. There are neither synthesis procedures nor deprotection conditions and enzymatic incorporation data disclosed for the nitrate group.

Buzby (US 2007-01171 04) discloses nucleoside triphosphates for SBS which are reversibly protected at the 3′-hydroxyl group and carry a label at the base. The label is connected via a cleavable linker such as a disulfide linker or a photocleavable linker. The linker consists of up to about 25 atoms. The 3′OR-protection group can be besides hydroxylamines, aldehydes, allylamines, alkenes, alkynes, alcohols, amines, aryls, esters, ethers, carbonitriles, phosphates, carbonates, carbamates, borates, sugars, phosphoramidates, phenylsulfanates, sulfates, sulfones and heterocycles also nitrates.

The methods disclosed herein can be used to sequence unknown or known nucleic acids or be used for genotyping. The methods can include the formation and use of a partially double-stranded circular DNA using synthetic hairpins and polymerase chain reaction amplicons. Both the antisense and sense strands can be maintained in a single circle so that both strands can be sequenced sequentially from the same concatemers formed via rolling circle amplification.

The methods and compostions disclosed herein can be used on next-generation sequencing (NGS) reactions that can be carried out using different primer sequences sequentially to amplify a plurality of concatemers as described herein. In some aspects, a sequencing reaction can be carried out using first a sequencing primer that can hybridize to a sequence within one of the hairpin adapters (e.g. a first or second primer binding site) followed by extension of the first primer and/or a one base addition-blocking step using, for example, dideoxynucleotides, can be used to block the sequencing fragments from further elongation. Next, sequencing can continue using a second sequencing primer that can hybridize to a sequence within the other hairpin adapter. Together, both strands (e.g., sense and antisense) can be sequenced from the same concatemer. In some aspects the concatemer can be attached to, for instance, a flow cell in the same position.

The method permits sequence information to be obtained and subsequently used to reconstruct large sequences, including the entire genome. Further, the method described herein can determine the sequences of both nucleic acid strands that can be located between to the two hairpin adapters.

In some aspects, the methods described herein can further include sequencing at least one of the plurality of concatamers.

Disclosed herein are methods of identifying at least one nucleotide of a sequence of interest, the method comprising: (a) forming a first partially double stranded circular DNA wherein the partially double stranded circular DNA contains (i) a sequence of interest, (ii) a first hairpin adaptor, and (iii) a second hairpin adaptor, wherein the first hairpin adaptor comprises a first primer binding site and the second hairpin adaptor comprises a second primer binding site; (b) amplifying the first partially double stranded circular DNA via rolling circle amplification, wherein amplification of the first partially double stranded circular DNA results in replicated strands, wherein during amplification at least one of the replicated strands is displaced from the first partially double stranded circular DNA by strand displacement replication, wherein the amplification of the first partially double stranded circular DNA results in a plurality of partially double stranded concatamers; (c) contacting at least one of the partially double stranded concatamers with a first primer, wherein the first primer hybridizes to the first or second primer binding, and (d) identifying at least one nucleotide of the sequence of interest adjacent or close to the first primer binding site.

In some aspects, the methods described herein can further comprise a step of contacting at least one of the partially double-stranded concatamers with a second primer. In some aspects, the second primer can hybridize to the first or second primer binding site. In some aspects, the first primer can hybridize to the first primer binding site and the second primer can bind to the second primer binding site. In some aspects, first primer can hybridize to the second primer binding site and the second primer can bind to the first primer binding site. In some aspects, the methods described herein can further comprise a step of identifying at least one nucleotide of the sequence of interest adjacent or close to the second primer binding site.

Disclosed herein are methods of identifying at least one nucleotide of a sequence of interest, the method comprising: (a) forming a first partially double stranded circular DNA wherein the partially double stranded circular DNA contains (i) a sequence of interest, (ii) a first hairpin adaptor, and (iii) a second hairpin adaptor, wherein the first hairpin adaptor comprises a first primer binding site and the second hairpin adaptor comprises a second primer binding site; (b) amplifying the first partially double stranded circular DNA via rolling circle amplification, wherein amplification of the first partially double stranded circular DNA results in replicated strands, wherein during amplification at least one of the replicated strands is displaced from the first partially double stranded circular DNA by strand displacement replication, wherein the amplification of the first partially double stranded circular DNA results in a plurality of partially double stranded concatamers, (c) contacting at least one of the partially double stranded concatamers with a first primer, wherein the first primer hybridizes to the first primer binding site, (d) extending the first primer in the presence of one or more dideoxynucleotides thereby generating a first primer elongation product, (e) contacting at least one of the partially double stranded concatamers with a second primer, wherein the second primer hybridizes to the second primer binding site; (f) extending the second primer, thereby generating a second primer elongation product; and (g) identifying at least one nucleotide of the sequence of interest adjacent or close to the first primer binding site and at least one nucleotide of the sequence of interest adjacent or close to the second primer binding site. In some aspects, elongation of the first primer can be terminated when the one or more dideoxynucleotides are incorporated into the first primer elongation product. In some aspects, the first primer elongation product is not removed prior to step (e). In some aspects, the first primer elongation product is not removed prior to step (g). In some aspects, neither the first nor the second primer elongation product is removed prior to step (g).

As described herein, a concatamer or plurality of concatamers disclosed herein can be used as a template for sequencing.

Disclosed herein are methods of identifying at least one nucleotide of a sequence of interest, the method comprising: (a) forming a first partially double stranded circular DNA wherein the partially double stranded circular DNA contains (i) a sequence of interest, (ii) a first hairpin adaptor, and (iii) a second hairpin adaptor, wherein the first hairpin adaptor comprises a first primer binding site and the second hairpin adaptor comprises a second primer binding site; (b) amplifying the first partially double stranded circular DNA via rolling circle amplification, wherein amplification of the first partially double stranded circular DNA results in replicated strands, wherein during amplification at least one of the replicated strands is displaced from the first partially double stranded circular DNA by strand displacement replication, wherein the amplification of the first partially double stranded circular DNA results in a plurality of partially double stranded concatamers, (c) contacting at least one of the partially double stranded concatamers with a first primer, wherein the first primer hybridizes to the first primer binding site, (d) extending the first primer in the presence of one or more dideoxynucleotides thereby generating a first primer elongation product, (e) contacting at least one of the partially double stranded concatamers with a second primer, wherein the second primer hybridizes to the second primer binding site; (f) extending the second primer, thereby generating a second primer elongation product; and (g) identifying at least one nucleotide of the sequence of interest adjacent or close to the first primer binding site and at least one nucleotide of the sequence of interest adjacent or close to the second primer binding site. In some aspects, the extension steps of (d) and (f) can be carried out using the well-known sequencing method referred to as the sequencing-by-synthesis (SBS) method. According to this method, nucleotide analogues can be used. In some aspects, the extension step of steps (d) and/or (f) can be carried out in the presence of one or more dideoxynucleotides. In some aspects, the extension step of steps (d) and/or (f) can be carried out in the presence of one or more nucleotide analogues that comprise a reversible 3′OH-protecting group. In some aspects the nucleotide analogues also comprise a unique label attached through a cleavable linker attached to the base of the nucleotide analogues. Examples of sequencing reactions and nucleotide analogues suitable for use in the disclosed methods of identifying at least one nucleotide of a sequence of interest can be found in: U.S. Pat. Nos. 6,664,079, 7,057,026, 7,345,159, 7,790,869, 8,088,575, 7,713,698, 7,635,578, 7,883,869, 8,298,792, 9,708,358, 9,719,139, 9,718,852 and application numbers PCT/US2007/024646, PCT/GB2002/005474, PCT/GB2003/003690, PCT/GB2003/003686, PCT/US16/60435, US 2017-0088891 A1, US 2017-0088575 A1, or US 2017-0088574 A1, all of which are incorporated by reference in their entirety for their teaching of nucleotide analogues, protecting groups, linkers, labels, sequencing reactions and methods of cleaving or uncapping the nucleotide analogues disclosed therein.

In some aspects, the nucleotide analogues comprise a 3′-OH position capped by a group comprising methylenedisulfide as a cleavable protecting group and a detectable label reversibly connected to the nucleobase of said nucleotide analogue. In some a nucleotide analogue with a reversible protecting group comprising methylenedisulfide and a cleavable oxymethylenedisulfide linker between the label and nucleobase can be used.

In some aspects, the step of extending the first primer in the presence of one or more dideoxynucleotides thereby generating a first primer elongation product can be performed in the presence of one or more of the nucleotide analogues or modified nucleotides described herein and one the sequencing and/or extension reactions is sought to be terminated, the modified nucleotide can simply not be exposed to an agent that otherwise would cleave the reversible 3′OH-protecting group. In some aspects, a nucleotide of a sequence of interest can be determined in an extension reaction of a first or second primer by determining the presence of a nucleotide analogue comprising a reversible 3′OH-protecting group. In some aspects, a nucleotide of a sequence of interest can be determined in an extension reaction of a first or second primer by determining the presence of a nucleotide analogues comprising a reversible 3′OH-protecting group and the reversible 3′OH-protecting group can be cleaved or removed from the nucleotide analogue, allowing for incorporation of another nucleotide analogue comprising a reversible 3′OH-protecting group and subsequent determination of the additional nucleotide analogue. In some aspects, the one-by-one extension of a first or second primer as described herein can be carried out for any number of cycles.

In some aspects, the step of extending the first primer in the presence of one or more dideoxynucleotides thereby generating a first primer elongation product can be performed in the presence of a dideoxynucleotides that once incorporated would irreversibly terminate the extension reaction. In some aspects, both modified nucleotides and dideoxynucleotides can be used in the extension reactions. The same reactions can be carried out with deoxynucleotides or modified deoxynucleotides in the step of extending the second primer, thereby generating a second primer elongation product.

In some aspects, one or more of four different nucleotide analogs can be added in steps (d) and/or (f), wherein each different nucleotide analogue comprises a different base selected from the group consisting of thymine or uracil or an analogue of thymine or uracil, adenine or an analogue of adenine, cytosine or an analogue of cytosine, and guanine or an analogue of guanine, and wherein each of the four different nucleotide analogues comprises a unique label.

In some aspects, steps (e) and (f) can be performed without removing the first primer or first primer elongation product. For example, after a first primer elongation product is generated, at least one of the partially double stranded concatamers can be contacted with a second primer and the second primer can be extended without removing or disassociating the first primer elongation product from the concatamer. In some aspects the blocking of all first primer elongation products generated can block any further elongation of these fragments (e.g. first primer elongation products) which could otherwise interfere during the elongation of the second primer. By not removing the first primer or first primer elongation products and therefore not submitting the concatemer to a heat dissociation step it allows the method to keep the concatemer in place and keep the registration or position on the surface where they are attached. The co-localization of both sequencing reads therefore allow for a paired-end approach. In other words the two strands of a concatamer can be sequenced sequentially without removing the first primer elongated during the extension of the first primer.

Disclosed herein are methods involving concatemer RCA-based NGS pair-end sequencing using a circular template that can be in the shape of a dumbbell. The methods disclosed herein can include the following steps. Circular templates for the RCA reaction can be generated by ligating two different palindromic hairpin adapters on each end of the double-stranded enriched targeted PCR library products. The key to pair-end sequencing is to use double-stranded DNA in order to include both sense (+) and antisense (−) strands in a single circle so that both strands can be sequenced sequentially from the same concatemers generated via RCA seeded onto a single flow cell. Once the hairpin adapters are ligated, they form a closed circular template which serves as a template for the RCA reaction resembling a “dumbbell.” The concatemers can be seeded onto the flow cell and NGS reaction can be performed using two different primers sequentially, initially with a sequencing primer from, for example, Adapter A and performing 50 cycles followed by a one base addition-blocking step using dideoxynucleotides to block the sequencing fragments from elongating any further. Adapter B, for example, can then used to perform another 50 cycles. This allows sequencing from both (+) and (−) strands from the same concatemer attached to the flow cell in the same position on a flow cell.

A single-stranded terminal overhang as described herein can allow the hairpin adaptors to control the direction of the sequencing step. In some aspects, the number of cycles for sequencing can be 20, 30, 40, 50, 60, 70, 80, 90, 100, 110, 120, 130, 140, 150, 160, 170, 180, 190, 200 and so on; and any number of cycles between. Generally, a single cycle incorporates a single nucleotide. The number of cycles can be higher or lower depending on amplicon length or equipment limitations. The number of cycles for sequencing can be monitored and regulated. In some aspects, the sequencing step can be terminated after a designated number of cycles. It is desirable to sequence the double-stranded portion of the amplicon and not the hairpin adaptor.

Disclosed herein are methods of identifying at least one nucleotide of a sequence of interest. In some aspects, the methods can comprise:
a) contacting at least one partially double stranded concatamer with a first primer, wherein the first primer hybridizes to a first primer binding site,
b) extending the first primer in the presence of one or more dideoxynucleotides thereby generating a first primer elongation product,
c) contacting at least one of the partially double stranded concatamers with a second primer, wherein the second primer hybridizes to a second primer binding site;
d) extending the second primer, thereby generating a second primer elongation product; and
e) identifying at least one nucleotide of the sequence of interest adjacent or close to the first primer binding site and at least one nucleotide of the sequence of interest adjacent or close to the second primer binding site.

In an aspect, prior to step b, the first primer can be extended in the absence of one or more dideoxynucleotides. In an aspect, the one or more dideoxynucleotides can be present during the entire extension step of step b. In an aspect, the one or more dideoxynucleotides can be added during the extension step of step b. In an aspect, the one or more dideoxynucleotides can be added after the first primer is extended in the absence of one or more dideoxynucleotides. In an aspect, the one or more dideoxynucleotides can be added after the first primer is extended for 20-200 cycles in the absence of one or more dideoxynucleotides. In an aspect, the partially double stranded concatamer can comprise a single stranded DNA portion and a double stranded DNA portion. In an aspect, the first and second primer binding sites can be located in a single stranded region of the partially double stranded concatamer. In an aspect, the first and second primer binding sites can be different. In an aspect, the single stranded DNA portion can be in a loop configuration. In an aspect, the partially double stranded region of the concatamer can contain a sequence of interest. In an aspect, the sequence of interest can be a human sequence of interest. In an aspect, the sequence of interest can be from genomic DNA. In an aspect, the extension of the first primer can be terminated when the one or more dideoxynucleotides are incorporated into the first primer elongation product. In an aspect, the first primer elongation product is not removed prior to step c. In an aspect, the first primer elongation product is not removed prior to step e. In an aspect, the second primer elongation product is not removed prior to step e. In an aspect, neither the first nor the second primer elongation product is removed prior to step e. In an aspect, the partially double stranded concatamer can be immobilized on a surface of a substrate. In an aspect, the one or more dideoxynucleotides can be added during the extension step of step d. In an aspect, the one or more dideoxynucleotides can be added after the second primer is extended in the absence of one or more dideoxynucleotides. In an aspect, the one or more dideoxynucleotides can be added after the second primer is extended for 20-200 cycles.

Disclosed herein are methods of identifying at least one nucleotide of a sequence of interest. In some aspects, the methods can comprise:
a) contacting at least one partially double stranded concatamer with a first primer, wherein the first primer hybridizes to a first primer binding site,
b) extending the first primer in the presence of one or more nucleotide analogues that comprise a 3′OH-protecting group thereby generating a first primer elongation product,
c) contacting at least one of the partially double stranded concatamers with a second primer, wherein the second primer hybridizes to a second primer binding site;
d) extending the second primer, thereby generating a second primer elongation product; and
e) identifying at least one nucleotide of the sequence of interest adjacent or close to the first primer binding site and at least one nucleotide of the sequence of interest adjacent or close to the second primer binding site.

In an aspect, prior to step b, the first primer can be extended in the absence of one or more nucleotide analogues. In an aspect, the one or more nucleotide analogues can be present during the entire extension step of step b. In an aspect, the one or more nucleotide analogues can be added during the extension step of step b. In an aspect, the one or more nucleotide analogues can be added after the first primer is extended in the absence of one or more dideoxynucleotides. In an aspect, the one or more nucleotide analogues can be added after the first primer is extended for 20-200 cycles in the absence of one or more dideoxynucleotides. In an aspect, the partially double stranded concatamer can comprise a single stranded DNA portion and a double stranded DNA portion. In an aspect, the first and second primer binding sites can be located in a single stranded region of the partially double stranded concatamer. In an aspect, the first and second primer binding sites can be different. In an aspect, the single stranded DNA portion can be in a loop configuration. In an aspect, the partially double stranded region of the concatamer can contain a sequence of interest. In an aspect, the sequence of interest can be a human sequence of interest. In an aspect, the sequence of interest can be from genomic DNA. In an aspect, elongation of the first primer can be terminated when the one or more nucleotide analogues are incorporated into the first primer elongation product. In an aspect, the first primer extension product is not removed prior to step c. In an aspect, the first primer elongation product is not removed prior to step e. In an aspect, the second primer elongation product is not removed prior to step e. In an aspect, neither the first nor the second primer elongation product is removed prior to step e. In an aspect, the partially double stranded concatamer can be immobilized on a surface of a substrate. In an aspect, the 3′OH-protecting group of the one or more nucleotide analogues can be a reversible 3′OH-protecting group. In an aspect, the 3′OH-protecting group of the one or more nucleotide analogues can be an irreversible 3′OH-protecting group. In an aspect, the one or more nucleotide analogues can further comprise a unique label attached through a cleavable linker attached to the base of the nucleotide analogues. In an aspect, the one or more nucleotide analogues can be added during the extension step of step d. In an aspect, the one or more nucleotide analogues can be added after the second primer is extended in the absence of one or more dideoxynucleotides. In aspect, one or more nucleotide analogues can be added after the second primer is extended for 20-200 cycles.

Disclosed herein are methods of identifying at least one nucleotide of a sequence of interest. In some aspects, the methods can comprise:
a) contacting at least one partially double stranded concatamer with a first primer, wherein the first primer hybridizes to a first primer binding site,
b) extending the first primer in the presence of a labeled nucleotide or nucleotide analogue thereby generating a first primer elongation product, and
c) identifying at least one nucleotide of a sequence of interest adjacent or close to the first primer binding site.

In an aspect, the method can further comprise adding one or more dideoxynucleotides or nucleotide analogues that comprise a 3′OH-protecting group, thereby terminating extension of the first primer elongation product. In an aspect, the method can further comprise:
d) contacting at least one of the partially double stranded concatamers with a second primer, wherein the second primer hybridizes to a second primer binding site and extending the second primer, thereby generating a second primer elongation product; and
e) identifying at least one nucleotide of a sequence of interest adjacent or close to the second primer binding site.

In an aspect, the one or more dideoxynucleotides or nucleotide analogues that can comprise a 3′OH-protecting group can be added after the first primer is extended for 20-200 cycles in step b. In an aspect, the partially double stranded concatamer can comprise a single stranded DNA portion and a double stranded DNA portion. In an aspect, the first and second primer binding sites can be located in a single stranded region of the partially double stranded concatamer. In an aspect, the first and second primer binding sites can be different. In an aspect, the single stranded DNA portion can be in a loop configuration. In aspect, the partially double stranded region of the concatamer can contain a sequence of interest. In an aspect, the sequence of interest can be a human sequence of interest. In an aspect, the sequence of interest can be from genomic DNA. In an aspect, the first primer elongation product is not removed prior to step d. In an aspect, the partially double stranded concatamer can be immobilized on a surface of a substrate. In an aspect, one or more dideoxynucleotides can be added during the extension step of step d. In an aspect, the labeled nucleotide or nucleotide analogue can comprise a reversible 3′OH-protecting group.

The methods described herein can also be used in connection with sequencing, SNP detection with single base extension of both strands juxtaposing the variant sequentially of immobilized rolonies (that can be in the shape of a dumbbell) attached to a solid support as well as any other methods of detecting or determining a sequence of interest.

### A. Fragmentation

Fragments can be derived from either an entire genome or from a selected subset of a genome. Many techniques are available for isolating or enriching fragments from a subset of a genome and are known to one of ordinary skill in the art.

In an aspect, shear forces during lysis and extraction of genomic DNA generate fragments in a desired range. In an aspect, methods of fragmentation include the use of restriction endonucleases.

In the case of mammalian sized genomes, fragmentation can be carried out in at least two stages, a first stage to generate a population of fragments in a size range of from about 100 kilobases (Kb) to about 250 Kb, and a second stage, applied separately to each 100-250 Kb fragment, to generate fragments in the size range of from about 50 to 600 nucleotides for generating concatemers for a random array. In an aspect, the fragments can be generated in the range of from about 300 to 600 nucleotides. In an aspect, the first stage of fragmentation can also be employed to select a predetermined subset of such fragments, e.g. fragments containing genes that encode proteins of a signal transduction pathway, and the like.

In an aspect, the sample genomic DNA can be fragmented using techniques known to one of ordinary skill in the art.

In an aspect, genomic DNA can be isolated as 30-300 kb sized fragments. Through proper dilution, a small subset of these fragments can be, at random, placed in discreet wells of multi-10 well plates or similar accessories. For example a plate with 96, 384 or 1536 wells can be used for these fragment subsets. One way to create these DNA aliquots can be to isolate the DNA with a method that naturally fragments to high molecular weight forms, dilute to 10-30 genome equivalents after quantitation, and then split the entire preparation into 384 wells. This can provide representation of all genomic sequences, and performing DNA isolation on 10-30 cells with 100% recovery efficiency assures that all chromosomal regions can be represented with the same coverage. By providing aliquots using this method, the probability of placing two overlapping fragments from the same region of a chromosome into the same plate well can be minimized. For diploid genomes represented with 10× coverage, there are 20 overlapping fragments on average to separate into distinct wells. If this sample can distributed over a 384 well plate, then each well can contain, on average, 1,562 fragments. By forming 384 fractions in a standard 384-well plate, there is about a 1/400 chance that two overlapping fragments can end up in the same well. Even if some matching fragments are placed in the same well, the other overlapping fragments from each chromosomal region provide the unique mapping information.

In an aspect, the prepared groups of long fragments can be further cut to the final fragment size of about 300 to 600 bases. To obtain sufficient (e.g., 10×) coverage of each fragment in a group, the DNA in each well can be amplified before final cutting using well-developed whole genome amplification methods.

All short fragments from one well can be then be arrayed and sequenced on one separate unit array or in one section of a larger continuous matrix. A composite array of 384 unit arrays can be used for parallel analysis of these groups of fragments. In the assembly of long sequences representing parental chromosomes, the algorithm can use the critical information that short fragments detected in one unit array belong to a limited number of longer continuous segments each representing a discreet portion of one chromosome. In almost all cases the homologous chromosomal segments can be analyzed on different unit arrays. Long (˜100 Kb) continuous initial segments form a tailing pattern and provide sufficient mapping information to assemble each parental chromosome separately as depicted below by relying on about 100 polymorphic sites per 100 kb of DNA.

In an aspect, amplification of the single targets obtained in the chromosomal separation procedure can be accomplished using methods known in the art for whole genome amplification. In an aspect, methods that produce 10-100 fold amplification can be used. In an aspect, these procedures do not discriminate in terms of the sequences that are to be amplified but instead amplify all sequences within a sample. Such a procedure does not require intact amplification of entire 100 kb fragments, and shorter fragments, such as fragments from 1-10 kb, can be used.

### B. Attachment of Hairpin Adapters

Disclosed herein are methods of producing a circular template for sequence analysis. In an aspect, the circular template can be a double-stranded circular DNA. In this method, a first partially double-stranded circular DNA can be formed. The method comprises combining the target nucleic acid molecule with a first primer set having a uracil at the 3′ end and incubating the target nucleic acid molecule with the first primer set under conditions that promote hybridization and replication of the target nucleic acid molecule, thereby producing a double-stranded amplicon, wherein the double-stranded amplicon comprises a first and second strand. The target nucleic acid molecule can be obtained by fragmentation of a larger piece of DNA, such as chromosomal or other genomic DNA.

Next, the double-stranded amplicon can be contacted or mixed with an enzyme to produce a first terminal overhang and a second terminal overhang at the 5′ end of the first strand and 5′ end of the second strand of the double-stranded amplicon. For example, the double-stranded amplicon can be contacted or mixed with an enzyme like USER which is a mixture of Uracil DNA glycosylase (UDG) and the DNA glycosylase-lyase Endonuclease VIII). UDG can catalyze the excision of the uracil base, forming an abasic (apyrimidinic) site while leaving the phosphodiester backbone intact and the nicked DNA can then generate sticky ends due to unstable double strand DNA portion upstream or downstream of the nick.

In some aspects, any known method can be used to generate the terminal overhang. In some aspects, the double-stranded amplicon can include one or more restriction sites. Restriction endonucleases recognition cites are described herein. Endonucleases recognize specific sequences of nucleotide base pairs within a double-stranded polynucleotide. Upon recognition of the specific sequence the endonuclease cleaves the polynucleotide sequence generating a single-stranded overhang of that sequence. The overhang can be a “sticky end.” In some aspects, each of the strands of the double-stranded amplicon can comprise the same Type II restriction endonuclease site. In other aspects, each of the strands of the double-stranded amplicon can comprise different Type II restriction endonuclease sites.

This step can be followed by mixing or contacting the double-stranded amplicon with the first hairpin adapter and the second hairpin adapter, wherein the first hairpin adapter comprises a first sequence complementary to the first terminal overhang the double-stranded amplicon and the second hairpin adapter comprises a second sequence complementary to the second terminal overhang at the 5′ end of the double-stranded amplicon and incubating the double-stranded amplicon with the first hairpin adapter and the second hairpin adapter under conditions that promote hybridization between the first hairpin adapter with the first terminal overhang of the double-stranded amplicon and the second hairpin adapter with the second terminal overhang of the double-stranded amplicon.

In some aspects, the hairpin adapters can include a 3′ overhang that is complementary to the adapter portion located upstream or downstream of the nick that is created after the Uracil excision and overhang creation on the amplicon see below:

In this aspect, the adapters (HR1 and HR2) are designed to generate hairpins that contain 3′ overhangs that are complementary to the adapter portion of the target amplicons to which they will be ligated. Each hairpin (i.e., HR1 and HR2) adapter has a specific 3′ overhang assuring the directionality of the binding to the amplicon. The amplicon adapters described herein can contain a uracil (U) at the 3′ end of the adapter. This uracil can be excised using a USER enzyme as described herein. The nick generated by this excision and removal due to instability of the small portion of DNA next to the nick generates amplicons with 5′ overhangs. These 5′ overhangs are complementary to the hairpin adapter 3′ overhangs.

Hairpin adapters with 5′ overhangs can be ligated to the 3′ overhangs of the amplicons using conventional techniques, one on each end of the amplicon. For example, the first hairpin adapter can be ligated to the first terminal overhang and the second hairpin adapter can be ligated to the second terminal overhang forming a closed circular template, wherein the closed circular template can be in a dumbbell configuration having a double-stranded portion (e.g., the linear portion) and two single stranded portions (e.g., the loop portion). Alternatively, the first hairpin adapter can be ligated to the first terminal overhang and the second hairpin adapter can be ligated to the second terminal overhang forming the partially double-stranded circular DNA.

In some aspects, the closed circular template can be generated without introducing uracils as described, but, rather using any method known to one of ordinary skill in the art. For example, introducing a restriction site into the double-stranded amplicon can also be used.

The closed circular template can be used to generate concatemers of the amplicon contained in the double-stranded circular DNA by rolling circle amplification, as described more fully below.

### C. Rolling Circle Amplification

The methods disclosed herein include preparing a target nucleic acid molecule for sequencing. The method can comprise forming a first partially double stranded circular DNA wherein the partially double stranded circular DNA contains (i) a sequence of interest having a first strand and a second strand, (ii) a first hairpin adapter, and (iii) a second hairpin adapter, wherein the first and second strands of the sequence of interest are complementary to each other and wherein the first hairpin adapter comprises a first primer binding site and the second hairpin adapter comprises a second primer binding site. This step can be followed by b) amplifying the first partially double stranded circular DNA via rolling circle amplification, wherein amplification of the first partially double stranded circular DNA results in replicated strands, wherein during amplification at least one of the replicated strands is displaced from the first partially double stranded circular DNA by strand displacement replication; wherein the amplification of the first partially double stranded circular DNA results in a plurality of concatemers.

In an aspect, the first hairpin adapter and the second hairpin adapter are different.

In an aspect, the first partially double stranded circular DNA can be in a dumbbell configuration having a double stranded portion flanked by two single stranded portions.

In an aspect, one of the two single-stranded portions can be adjacent to each end of the double stranded portion.

In an aspect, one of the two single-stranded portions comprises part of the first hairpin adapter and one of the two single-stranded portions can comprise the second hairpin adapter.

In an aspect, each of the concatamers comprises a single-stranded DNA portion and a double-stranded DNA portion.

In an aspect, the single-stranded DNA portion can be in a loop configuration.

In an aspect, each of the concatamers can have a copy of the sequence of interest and a copy of at least one of the hairpin adapters.

In an aspect, each of the concatemers can have a copy of the sequence of interest and a copy of both of the hairpin adapters.

In an aspect, the concatemers can comprise, in order, a portion of one of the hairpin adapters, a copy of the first strand of the sequence of interest, a copy of one of the hairpin adapters, and a copy of the second strand of the sequence of interest, wherein the first and second strands of the sequence of interest can be hybridized together. In an aspect, the copies of the hairpin adapters can be different. In an aspect, the portion of the one of the hairpin adapters can be a portion of the first hairpin adapter. In an aspect, the portion of the one of the hairpin adapters can be a portion of the second hairpin adapter.

In an aspect, the concatemers can comprise, in order, a copy of one of the hairpin adapters, a copy of the first strand of the sequence of interest, a copy of one of the hairpin adapters, and a copy of the second strand of the sequence of interest, wherein the first and second strands of the sequence of interest can be hybridized together. In an aspect, the copies of the hairpin adapters can be different.

In an aspect, the sequence of interest can be a human sequence of interest. In an aspect, the sequence of interest can be from genomic DNA. In an aspect, the target nucleic acid molecule can be double-stranded DNA. In an aspect, first hairpin adapter comprises a first restriction enzyme binding site. In an aspect, the first hairpin adapter can be partially single-stranded, wherein the single-stranded portion comprises an overhang portion complementary to one terminus of the sequence of interest. In an aspect, the second hairpin adapter comprises a second restriction enzyme binding site. In an aspect, the second hairpin adapter can be partially single-stranded, wherein the single-stranded portion comprises an overhang portion complementary to one terminus of the sequence of interest.

Many different forms of RCA can be used in the disclosed method, most of which are described in U.S. Pat. No. 5,854,033 and WO 97/19193. For example, linear rolling circle amplification (LRCA) involves the basic rolling circle replication of an amplification target circle to form a strand of TS-DNA. Exponential rolling circle amplification (ERCA) involves replication of TS-DNA by strand displacement replication initiated at the numerous repeated sequences in the TS-DNA. Multiple priming on both strands of TS-DNA leads to an exponential amplification of sequences in the amplification target circle. Poly-primed rolling circle amplification (PPRCA) provides greatly increased amplification due to secondary, tertiary, quaternary, and higher order amplification processes occurring from a primary tandem sequence (TS-DNA) product (described in U.S. Pat. No. 6,291,187). If desired, the TS-DNA can be collapsed into a compact structure for detection as described in WO 97/19193.

### D. Methods of Generating Concatemers

In an aspect, the target nucleic acid molecules can be generated using a rolling circle amplification reaction. The method can be used to amplify one or more specific sequences, an entire genome or other DNA of high complexity. Guidelines for selecting the conditions and reactions for said reaction is known to one of ordinary skill in the art. Generally, rolling circle amplification can be carried out using partially double-stranded circular DNA as described herein. Additional components used in a rolling circle amplification include a set of primers, a DNA polymerase, nucleoside triphosphates and a polymerase reaction buffer. The components can be combined, for example, such that a set of primers can be mixed with a partially double-stranded circular DNA sample, producing a primer-partially double-stranded circular DNA sample mixture under conditions that promote hybridization between the primers and the target sequence in the primer-partially double-stranded circular DNA mixture. Next, the DNA polymerase can be mixed with the primer-partially double-stranded circular DNA mixture to produce a polymerase-partially double-stranded circular DNA mixture, followed by incubating the polymerase-partially double-stranded circular DNA mixture under conditions that promote replication of the target sequence. Strand displacement can be accomplished by using a strand displacing DNA polymerase or a DNA polymerase in combination with a compatible strand displacement factor. In an aspect, the primers anneal to the DNA circles and the DNA polymerase has strand displacement activity such that it extends the 3′ ends of the primers annealed to the DNA circles thereby forming concatemers of DNA circle complements. The following is an example of a rolling circle amplification reaction. In a 100 μL reaction mixture, the following components can be combined:

| Component | Volume [μl] | Final concentration |
|---|---|---|
| Nuclease free water | x | |
| Phi29 Pol 10x reaction | 20 | 1x (Enzymatic) |
| 1 mM AA-dUTP | 1 | 10 μM |
| 10 mM dNTP mix | 4 | 1 mM each |
| Rolony primer R (2 μM) | 1 | 0.02 μM |
| Circularized library | x | 0.1-5 pmol of |
| ss DNA | | 200 nt circle |
| | 97 μl | |

0.1-5 pmol circular DNA, 0.01 units/4 phage ϕ29 DNA polymerase, 1 μl of 10 U/μl Phi 29 DNA polymerase (Enzymatics) and 2 μl of 0.1 U/μl Inorganic Pyrophosphatase (New England Biolab) 1 mM dNTP, 1×ϕ29 DNA polymerase reaction buffer. The RCA reaction can be carried out at 30° C. for 4 hours.

Concatemers produced by rolling circle amplification can be approximately uniform in size; accordingly, in some aspects, methods of making arrays as described herein can include a step of size-selecting concatemers. For example, in one aspect, concatemers can be selected as a population that has a coefficient of variation in molecular weight of less than about 30%; and in another aspect, less than about 20%. In one aspect, size uniformity can be further improved by adding low concentrations of chain terminators, such ddNTPs, to the rolling circle amplification reaction mixture to reduce the presence of very large concatemers, e.g. produced by DNA circles that are synthesized at a higher rate by polymerases. In an aspect, concentrations of ddNTPs can be used that result in an expected concatemer size in the range of from 50-250 Kb, or in the range of from 50-100 Kb. In another aspect, concatemers can be enriched for a particular size range using conventional separation techniques, e.g. size-exclusion chromatography, membrane filtration, or the like.

Using the rolling circle amplification system, the desired DNA fragment can be “cloned” into a DNA hairpin adapter and replicated by linear concatemerization. The target nucleic acid molecule can immediately be in a form suitable for hybridization and enzymatic methodologies without the need to passage through bacteria.

The rolling circle amplification process relies upon the desired target molecule first being formed into a circular substrate. This linear amplification can use the original DNA molecule, not copies of a copy, thus, ensuring fidelity of sequence. As a circular entity, the molecule can act as an endless template for a strand displacing polymerase that extends a primer complementary to a portion of the circle. The continuous strand extension creates long, single-stranded DNA consisting of hundreds of concatemers comprising multiple copies of sequences complementary to the circle.

### E. Methods of Creating Arrays

In an aspect, arrays can be generated on a solid surface for sequence analysis. In an aspect, an array comprises beads containing sequences for further analysis wherein the location of the beads can be random. In some aspects, the location of the beads can have a predetermined pattern of binding sites. Capture probes can be attached to the glass surface to maintain amplified molecules for hybridization. Capture probe molecules can be spaced such that they are able to keep concatenated copies of a target molecule tightly bound to a glass surface area. Glass activation chemistry can be used to create a monolayer of isothiocynaate reactive groups for attaching amine modified capture oligonucleotides.

The immobilized target molecules can form a high density array. The densities of single molecules can be selected that permit at least twenty percent, at least thirty percent, at least forty percent or at least a majority of the molecules to be resolved individually by the signal generation and detection systems used. In an aspect, single-molecule arrays comprising nucleic acid molecules can be individually resolvable by optical means and sequencing can be carried out using the said arrays. In an aspect, single molecule array-based sequencing methods can be used with molecule-specific probes having fluorescent labels. A density can be selected such that at least a majority of single molecules have a nearest neighbor of 200 nm or greater.

In an aspect, selecting densities of randomly placed single molecules can be carried out by providing, on a surface, discrete spaced apart regions that are substantially the sole sites for attaching single molecules. For example, concatemers can be positioned such that they do not bind to the regions in between the attached single molecules. Generally, the area of discrete spaced apart regions can be selected to correspond to the size of single molecules disclosed herein so that when the single molecules are applied to the surface substantially every region can be occupied by no more than one single molecule. Thus, a single molecule can cover all linkages to the surface at a particular discrete spaced apart region thereby reducing the chance that a second single molecule will also bind to the same region. In an aspect, substantially all of the capture oligonucleotides in a discrete spaced apart region hybridize to adapter oligonucleotides in a single macromolecular structure. The length and sequence(s) of capture oligonucleotides can vary. In an aspect, the length of the capture oligonucleotide can range from about 8 to 30 nucleotides and its selection is with the capability of one of ordinary skill in the art. In an aspect, the discrete spaced apart regions can be less than 1 μm². In another aspect, the discrete spaced apart regions can be in the range of from 0.04 μm² to 1 μm².

In an aspect, photolithography, electron beam lithography, nano imprint lithography and nano printing can be used to generate patterns on a variety of surfaces.

In an aspect, high density structured random DNA array chips can have capture oligonucleotides concentrated in small, segregated capture cells aligned into a rectangular grid formation. Each capture molecule can bind one copy of the matching adapter sequence on the RCA produced concatemer. Methods of generating a patterned DNA chip are known in the art.

RCA “molecular cloning” allows the application of the saturation/exclusion (single occupancy) principle in making random arrays. The exclusion process is not feasible in making single molecule arrays if an in situ amplification is alternatively applied. RCA concatemers provide an optimal size to form small non-mixed DNA spots. Each concatemer of about 100 kb can occupy a space, for example, of about 0.1×0.1×0.1 μm, so that the RCA products can fit into 100 nm capture cells. An advantage of the RCA products is that the single-stranded DNA can be ready for hybridization and can be flexible for forming a randomly coiled ball of DNA.

A variety of chemical modifications can be used to alter surface properties, increasing the compatibility of the master mold with a wide range of materials, thus allowing the use of a small feature, low-density mold to create high density arrays. In an aspect, a mold with a 4 μm feature pitch can be used to create a one μm feature pitch on the substrate by printing the same substrate 16 times in a 4 by 4 grid.

In an aspect, a method of creating DNA arrays comprises using a thin layer of photo-resist to protect portions of the substrate surface during the functionalization process. The patterned photo-resist can be removed after functionalization, leaving an array of activated areas. Another approach comprises attaching a monolayer of modified oligonucleotides to the substrate. The oligonucleotides can be modified with a photocleavable protecting group. These protecting groups can be removed by exposure to an illumination source, allowing patterned ligation of a capture oligonucleotide for attachment concatemers by hybridization.

In an aspect, a commercially available, optically flat, quartz wafer can be spin coated with a 100-500 nm thick layer of photo-resist. The photo-resist can be baked on to the quartz wafer, and an image of a reticle with a pattern of spots to be activated can be projected onto the surface of the photo-resist, using a stepper. After exposure, the photo-resist can be developed, removing the areas of the projected pattern which were exposed to the UV source. This is accomplished by plasma etching, a dry developing technique capable of producing fine detail. The wafer can then be baked to strengthen the remaining photo-resist. After baking, the quartz wafer is ready for functionalization.

### F. Methods of Making Replica Arrays

In an aspect of the methods disclosed herein, complementary polynucleotides can be synthesized on a master array and then transferred to a replica array. To carry out the transfer, two surfaces can be contacted in the presence of heating to denature double-stranded DNA and the free, newly made DNA strands. In another aspect, the transfer can be carried out by applying an electric field to discriminatively transfer the replicated DNA that has about 5-50 times more charge than the primers. In a further aspect, after hybridizing the transferred strand, a reverse field can be combined with a reduction in temperature to move the primers back to the master array. When the transfer is carried out using an electric field, porous glass can be used to allow the application of the electric field.

In an aspect, a capture oligonucleotide can be designed to correspond to the end of an amplicon opposite to the priming site to assure exclusive retention of the full length copies. In an aspect, multiple transfers to the same replica can be completed to generate a stronger signal.

In an aspect, the substrate for the replica array contains primers for initiating DNA synthesis using template DNA attached on the first array. After contacting surfaces of the master array and support of the “to be formed” replica array in the presence of DNA polymerase, dNTPs and suitable buffer at optimum temperature, primer molecules hybridize to the template DNA on the master array and become extended by the polymerase. A stopping agent such as double-stranded DNA can be used to stop DNA at the end of one copy. By increasing temperature, or by using other DNA denaturing agents, DNA strands can separate and the replica array can be separated form the first array. To prevent removal of original DNA from the master array, the original DNA can be directly (or indirectly via capture oligonucleotide) covalently attached to the master array support.

In an aspect, primers can cover the substrate surface for array preparation. A primer density of 10,000 per μm² can provide a local concentration in one micron between two supports. Primers can have long attachment linkers to reach to the DNA template on the first array's support. A flat surface can be used to assure close proximity of the two surfaces.

Replica arrays can be used to generate additional replicas.

Replica arrays can also be used for parallel analysis of the same set of DNA fragments such as hybridization with a large number of probes or probe pools.

### G. Oligonucleotide Synthesis

Rolling circle replication primers, detection probes, address probes, amplification target circles, DNA strand displacement primers, and any other oligonucleotides can be synthesized using established oligonucleotide synthesis methods. Methods to produce or synthesize oligonucleotides are well known in the art. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6; Wu et al, Methods in Gene Biotechnology (CRC Press, New York, N.Y., 1997), and Recombinant Gene Expression Protocols, in Methods in Molecular Biology, Vol. 62, (Tuan, ed., Humana Press, Totowa, N.J., 1997)) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or PerSeptive Expedite). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

Many of the oligonucleotides described herein can be designed to be complementary to certain portions of other oligonucleotides or nucleic acids such that stable hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

### Kits

Described herein are kits that can be used for preparing arrays (e.g., random arrays) as described herein and for using the same for various applications. Kits for applications of random arrays include, but are not limited to, kits for determining the nucleotide sequence of target polynucleotides. In an aspect, the kits comprise at least one support having a surface and one or more reagents useful for constructing a random array as disclosed herein or for carrying out an application therewith. Such reagents include, without limitation, nucleic acid primers, probes, hairpin adapters, enzymes, and the like, and can be each packaged in a container, such as, without limitation, a vial, tube or bottle, in a package suitable for commercial distribution, such as, without limitation, a box, a sealed pouch, a blister pack and a carton.

In an aspect, the package can contain a label or packaging insert indicating the uses of the packaged materials. As used herein, “packaging materials” can include any article used in the packaging for distribution of reagents in a kit, including without limitation containers, vials, tubes, bottles, pouches, blister packaging, labels, tags, instruction sheets and package inserts.

In an aspect, the kits disclosed herein provide for sequencing a target polynucleotide comprising the following components: (i) a support having a planar surface having an array of optically resolvable discrete spaced apart regions, wherein each discrete spaced apart region has an area of less than 1 μm²; (ii) a first set of probes for hybridizing to a plurality of concatemers randomly disposed on the discrete spaced apart regions, the concatemers each containing multiple copies of a DNA fragment of the target polynucleotide; and (iii) a second set of probes for hybridizing to the plurality of concatemers such that whenever a probe from the first set hybridizes contiguously to a probe from the second set, the probes are ligated. Such kits can further include a ligase, a ligase buffer, and a hybridization buffer. In some aspects, the discrete spaced apart regions can have capture oligonucleotides attached and the concatemers can each have a region complementary to the capture oligonucleotides such that said concatemers can be capable of being attached to the discrete spaced apart regions by formation of complexes between the capture oligonucleotides and the complementary regions of said concatemers.

In an aspect, the kits are provided for circularizing nucleic acid fragments. In an aspect, kits can include the following components: (a) two hairpin adapters for ligating to the nucleic acid fragments and forming DNA circles therewith (b) a terminal transferase for attaching a homopolymer tail to said DNA fragments to provide a binding site for a first end of said adapter oligonucleotide, (c) a ligase for ligating a strand of said hairpin adaptor to ends of said nucleic acid fragments to form said nucleic acid circle, (d) a set of primers for annealing to a region of the strand of said hairpin adapters, and (e) a DNA polymerase for extending the primer annealed to the strand in a rolling circle amplification reaction. In a further aspect, the above hairpin adapter can have a second end having a number of degenerate bases in the range of from 4 to 12. The kit disclosed herein can further include reaction buffers for the terminal transferase, ligase, and DNA polymerase.

In an aspect, the kits for circularizing DNA fragments can use a CircLigase™ enzyme (Epicentre Biotechnologies, Madison, Wis.). The kit comprises a volume exclusion polymer. In a further aspect, the kit can include the following components: (a) reaction buffer for controlling pH and providing an optimized salt composition for CircLigase, and (b) CircLigase co-factors. In another aspect, a reaction buffer for the kits comprises 0.5M MOPS (pH 7.5), 0.1 M KCl, 50 mM MgCl2, and 10 mM DTT. In another aspect, the kits can further include CircLigase™, e.g. 10-100 μL CircLigase solution (at 100 unit/μL). Suitable volume exclusion polymers include polyethylene glycol, polyvinylpyrrolidone, dextran sulfate, and like polymers. In one aspect, polyethylene glycol (PEG) can be 50% PEG4000. In an aspect, a kit for circle formation includes the following: reaction buffer, 10×5 μl, water 21.7 μl, and T4 DNA Ligase 1 μl (1 unit), for a total of 30 μl.

The above components can be used in a number of different protocols known in the art, for example: (1) Heat DNA at 60-96° C. depending on the length of the DNA (single-stranded DNA templates that have a 5′-phosphate and a 3′-hydroxyl group); (2) Preheat 2.2× reaction mix at 60° C., for about 5-10 min; (3) if DNA was preheated to 96° C., cool it down at 60° C. or mix DNA and buffer at 60° C. without cooling it down and incubate for 2-3 h; and (4) heat-inactivate enzyme to stop the ligation reaction.

### EXAMPLES

### Example 1: Paired-End Sequencing Using Concatemers: Dumbbell or Circular Template Approach

An example of the methods disclosed herein can be referred to as the “dumbbell approach” or circular template approach. This approach can be a simple method of performing paired-end sequencing using concatemers. A circle can be formed by the ligation of two hairpin structures (containing large loops) to a PCR amplicon. The PCR product can then be amplified with uracil-containing primers that can be cleaved by a User™ enzyme to generate cohesive ends that can be ligated to the two hairpin structures in a specific orientation.

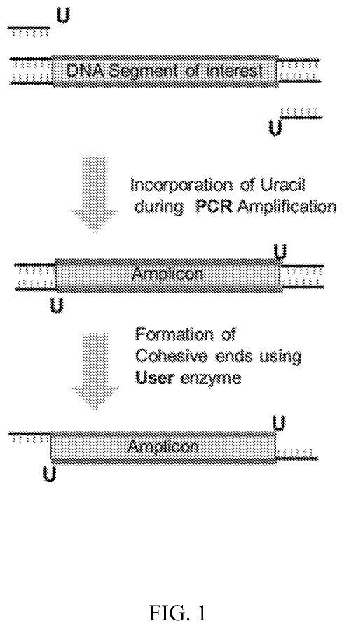

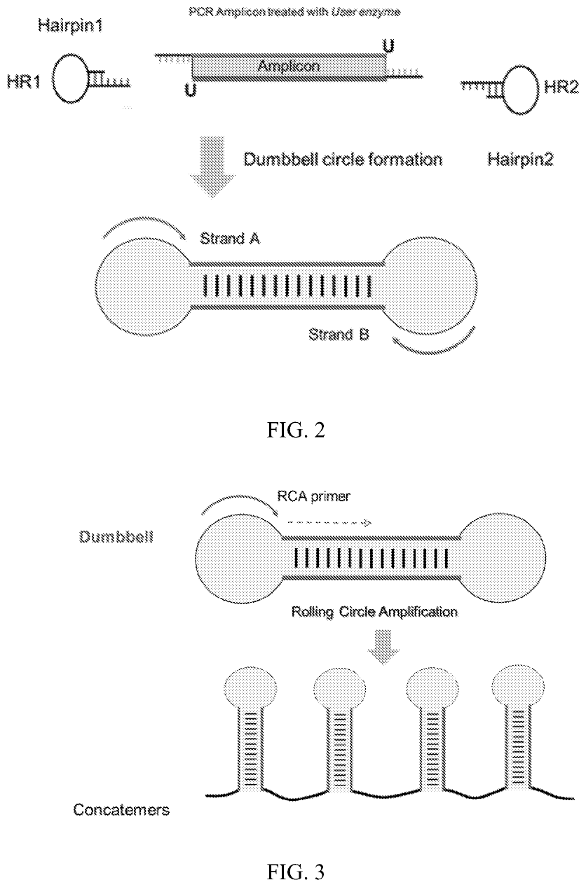

First, DNA fragments of interest are amplified via PCR using uracil-containing primers. The amplified fragment (e.g., amplicon) contains two uracils juxtaposing the amplicon sequence. The amplicons are cleaved by the User™ enzyme (FIG. 1) to generate cohesive ends that can be ligated, in a specific orientation, to two hairpin structures (see, FIG. 2).

Below shows the hairpin structure (SEQ ID NO: 8, top; SEQ ID NO: 9, bottom):

Next, double-stranded DNA is used to include sense (+) and antisense (−) strands in a single circle; both strands can be sequenced sequentially from the same RCA-produced concatemers seeded onto a single flow cell. The double-stranded amplicons with cohesive ends are ligated to two hairpin structures (HR1 and HR2, see FIG. 2). Once the adapters were ligated, a closed circular was formed serving as a template for the RCA reaction. The closed circle has a dumbbell appearance (see, dumbbell circle formation in FIG. 2). The closed circle can be referred to as “the circular template.”

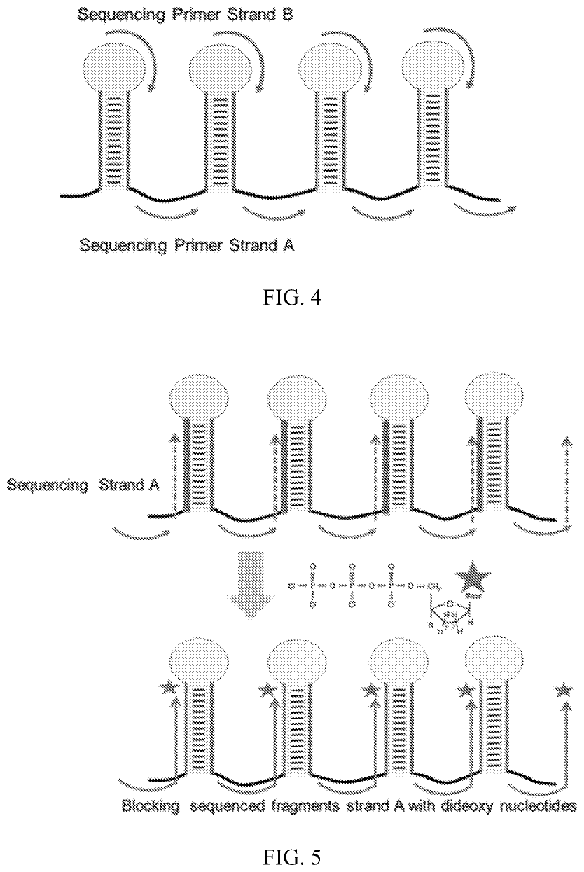

The two hairpin structures upon ligation can be used as circular template for RCA using Phi 29 enzyme. The products generated are concatemers of the amplicon contained in the circular template (FIG. 3). The concatemers can be sequenced using two primers (e.g., sequential sequencing; see, FIG. 4). One primer allows for the sequencing of strand A and one primer can be used for the sequencing strand B. Next, the concatemers are seeded onto a flow cell and a sequencing reaction (e.g., NGS) can be performed using two different primers sequentially, initially with a sequencing primer from Adapter A to sequence strand A and performing 50-150 cycles (see, FIG. 5).

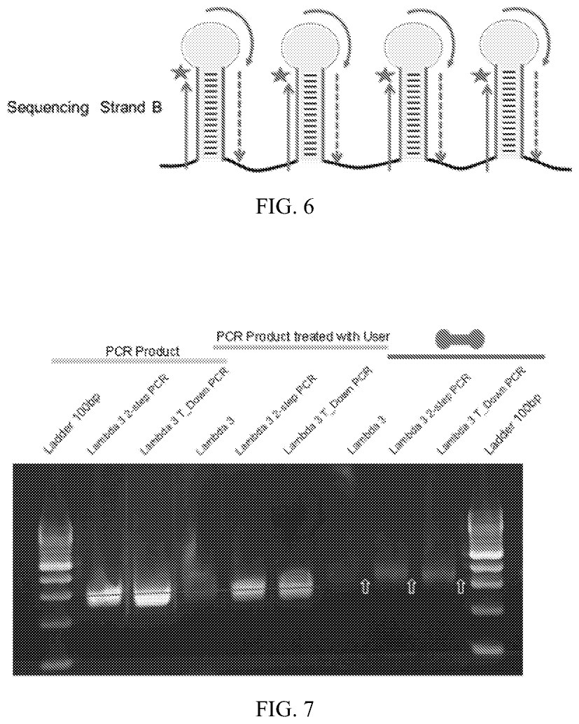

The sequencing of strand A can be followed by a one base addition-blocking step using dideoxynucleotides to impair the sequencing fragments generated from elongating any further during the second strand sequencing. Adapter B sequencing primers are used to perform another 50-150 cycles. This allows sequencing of the other strand from the same concatemer attached to the flow cell in the same position on a flow cell (see, FIG. 6).

Materials and Methods

Three lambda clones (13, 36 and 42) from GeneWiz were tested corresponding to SEQ ID NOs: 1, 2 and 3). Their sequences are shown in Table 1. Underscore nucleotides indicate the adapter portions complementary to M13 sequence or B2 sequence, the M13 being the shortest of the two.

TABLE 1
Lamda clones.

| | | SEQ |
|---|---|---|
| | | ID |
| Clone | Sequence | NO: |
| 13 | ACTTCAATTTACTATGTAGCAAAGGATACTCCGACGCGGCC | 1 |
| | GCAGCATATAGCCTGGTGGTTCAGGCGGCGCATTTTTATTG | |
| | CTGTGTTGCGCTGTAATTCTTCTATTTCTGATGCTGAATCA | |
| | ATGATGTCTGCCATCTTTCATTAATCCCTGAACTGTTGGTT | |
| | AATACGCTTGAGGGTGAATGCGAATAATAAAAAAGGAGCCT | |
| | GTAGCTCCCTGATGATTTTGCTTTTCATGTTCATCGTTCCT | |
| | TAAAAGACGCAGTTTAACACTGGCCGTCGTTTTACA | |
| 13 | TAAAACGACGGCCAGTGAATGCAAAGAAGATAACCGCTTC | 2 |
| | CGACCAAATCAACCTTACTGGAATCGATGGTGTCTCCGGTG | |
| | TGAAAGAACACCAACAGGGGTGTTACCACTACCGCAGGAA | |
| | AAGGAGGACGTGTGGCGAGACAGCGACGAAGTATCACCGA | |
| | CATAATCTGCGAAAACTGCAAATACCTTCCAACGAAACGCA | |
| | CCAGGCTGCGGCCGCGTCGGAGTATCCTTTGCTACATAGTA | |
| | AATTGAAGT | |
| 42 | GTAAAACGACGGCCAGTCTGCGATTCTCACCAATAAAAAAC | 3 |
| | GCCCGGCGGCAACCGAGCGTTCTGAACAAATCCAGATGGA | |
| | GTTCTGAGGTCATTACTGGATCTATCAACAGGAGTCATTAT | |
| | GACAAATACAGCAAAAATACTCAACTTCGGCAGAGGTAACT | |
| | TTGCCGGACAGGAGCGTAATGTGGCAGATCTCGATGATGGT | |
| | TACGCCAGACTATCAAATATGCTGCTTGAGCTGCGGCCGCG | |
| | TCGGAGTATCCTTTGCTACATAGTAAATTGAAGT | |

PCR conditions with Uracil-containing primers contained: Uracil-containing B2 forward (10 uM), 2.5 μl; Uracil-containing M13 (10 uM) M13, 2.5 μl; 10× hot start Taq reaction buffer, 5 μl; 10 mM dNTPs, 1 μl; lambda clones #13, 36, 42, (separately), 1 ng (1 μl); hot start taq DNA Polymerase (2 U/μl); 0.5 μl; and nuclease free H₂O, 37.5 μl. The following PCR temperature steps were carried out: 95° C., 4 min; 94° C., 1 min; 75° C., 1 min, −1° C. each cycle, 15×; 68° C., 2 min; 94° C., 30 sec; 57° C., 30 sec, 30×; 72° C., 1 min; 72° C., 5 min; and 4° C., end.

The PCR primer containing uracil for paired end clone library construction included (SEQ ID NO: 10, top; SEQ ID NO: 11, bottom):

Pho5 ′TAGCAAC [symbol] GTA A AA CGA CGG CCA GT M13_
Adapter
Pho5′ ACT TCA [symbol] T TAC TAT GTA GCA AAGG′3 B2
Primer

The User™ enzyme treatment included the following: Clone 13, 36 and 42 double-stranded ligation product (GeneWiz); Cutsmart buffer (B7204S, NEB); User™ enzyme (M5505S, NEB), 2.2% agarose Lonza flash gel (Lonza); and Qiaquick PCR purification kit (Qiagen).

Components used in hairpin ligation included: Clone 13, 36 and 42 double-stranded ligation product (GeneWiz); T4 ligase buffer (NEB); T4 ligase enzyme (NEB); 2.2% agarose Lonza flash gel (Lonza) and Qiaquick PCR purification kit (Qiagen).

The hairpin structure and sequence is shown below:

The sequences underlined above hybridize to close the hairpin structure. The short sequences following the sequences in underline above, for example, TAGCAACT (SEQ ID NO: 4, 5′ to 3′) and ACTTCAAT (SEQ ID NO: 5, 5′ to 3′) correspond to the overhang portion that hybridizes to the amplicon. The sequences in the box comprise the loop portion of the hairpin structure. These sequences are complementary to the primer (e.g., sequencing primer).

The rolling circle amplification reaction included the following components:

| Component | Volume | Final concentration |
|---|---|---|
| Nuclease free water | 76 | |
| Enzymatics 10x reaction buffer | 10 | 1x (Enzymatic) |
| 1 mM AA-dUTP | 1 | 10 μM |
| 10 mM dNTP mix | 4 | 1 mM each |
| Concatemer primer (2 μM) | 1 | 0.02 μM |
| Circularized DNA (20 ng/μL) | x | Lambda total 1 pmol |
| RecA | 8 μL | 0.08 μg/uL |
| TOTAL | 100 μl | |

RCA was performed at 30° C. for four hours in the presence of Phi 29 enzyme.

The concatemer (e.g., rolony) primer sequence is shown below (SEQ ID NO: 12, top; SEQ ID NO: 15, bottom):

The box contains the sequence of the hairpin loop, the concatemer primer sequence and also the sequence that is used to hybridize the second primer during the sequencing of the second strand.

The concatemers were seeded on a flow cell using the following components:

| Component | Final cone |
|---|---|
| Concatemer DNA Sample | 43.5 (50-100 ng/μL) |
| Reference Beads (Optional) | 0.2 million/μL |
| 1-ethyl-3-(3-dimethylaminopropyl) | 2.5 μL of 300 mg/mL |
| carbodiimide hydrochloride (EDC) | |
| N-Hydroxy Succinimide (NHS) | 2.5 μL of 150 mg/mL |
| Bis-SulfoSuccinimidyl | 1.3 μL of 2 mg |
| Suberate (B53) | pellet/140 μL |
| TOTAL | 50 μL |

| Component | Final concentration |
|---|---|
| Wash Buffer A [WB11] | 3 μL |
| FWR or REV Seq Primer (100 μM) | 1.5 μL of 250 μM |
| H2O | 25.5 μL H2O |
| TOTAL | 30 μL |

A library of hairpin adapters was constructed:

Library Construction Hairpin (Dumbbell Approach) _Large Loop (New Sequence Loop)

Sequences from top to bottom: SEQ ID NO: 12, SEQ ID NO: 15, SEQ ID NO: 15, SEQ ID NO: 16, SEQ ID NO: 13, and SEQ ID NO: 14.

Results

Lambda clones were prepared showing the elimination of PCR primer dimer products using touch-down PCR or 2-step PCR. The product was treated with User™ to generate cohesive ends that allow the ligation of two hairpin adapters in a specific orientation. After ligation, the PCR amplicons generate a circular template as shown by an upward shift (arrows) on the gel (see, FIG. 7).

The circular template was used to generate concatemers. The concatemers (e.g., rolony nanoballs) were seeded onto the flow cell and Next-generation sequencing reaction was performed sequentially using two different primers, initially with a sequencing primer from Adapter A and performing 50 cycles followed by a one base addition-blocking step using dideoxynucleotides to impair the first strand sequencing fragments from elongating any further. Adapter B was then used to perform another 50 cycles. This allowed sequencing from both (+) and (−) strand from the same concatemer attached to the flow cell in the same position on the flow cell.

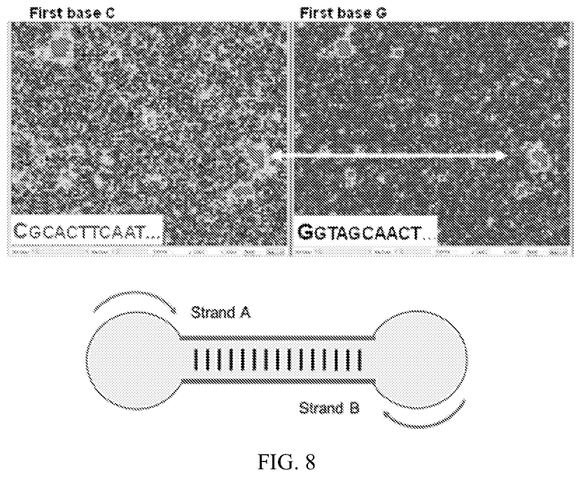

After sequencing, the images were studied manually and visually. The (+) and (−) strands were aligned for a couple of cycles. The first base read was not the same (see, FIG. 8). Different first base calling can be expected when different sequencing primers are used sequentially after a blocking step.

The signals were observed for the first base on the same flowcell for forward (left) and reverse strand (right) (50 cycles) after a ddNTP blocking step. The results indicate that the concatemer aligned and that the sequence changed depending on which sequencing primers were used forward (+) or reverse strand (−).

### Example 2: Sequence Analysis

The sequences generated from both strands were analyzed. Sequence occurrences were generated and clones were identified using the reverse strand located closest to the end of the adapter sequence. Therefore, during a 50 cycle run, the amplicon segment was read. This was not the case for the forward strand where the adapter was longer and, therefore, longer cycles would be required to reach the amplicon segments.

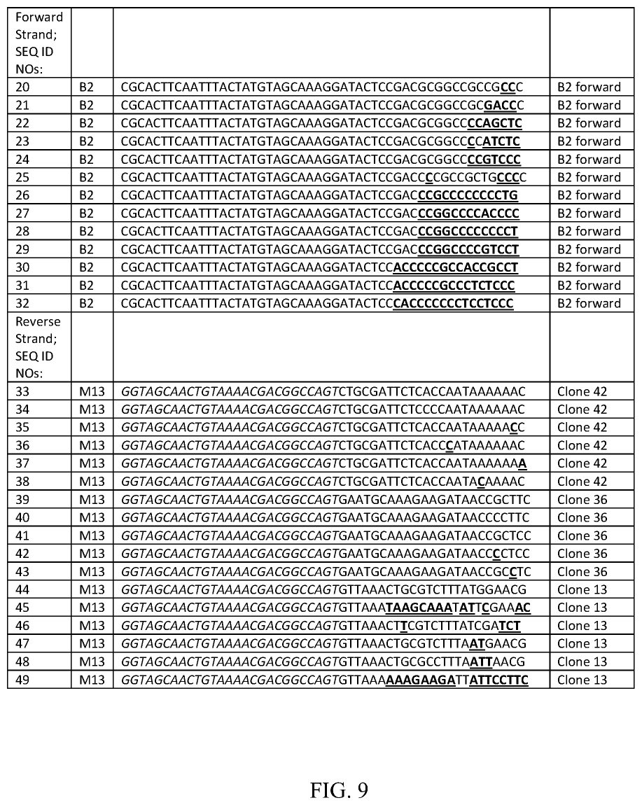

FIG. 9 shows an example of the sequence occurrences obtained using sequential paired-end sequencing (SPAIRS. Lambda-3 (clones 13, 36 and 42) were used. The reads obtained by SPAIRS is described below. The amount is subdivided by the clones which can be distinguished using the reverse strand sequence occurrences within the first 50 cycles (M13 adapter sequence is 27 nt). Twenty-three nt can be used for clone identification. Each of the clones were represented in the sequences generated. The M13 adapter sequence is shown in magenta (see, FIG. 9). The clones could not be distinguished using the forward strand for the first 50 cycles because the adapter is 50 nucleotides long. The number of cycles correspond to the number of nucleotides (e.g., bases) that were sequences in both directions. Each cycle corresponds to the sequence-by-synthesis steps that take place during the sequencing, for instance, on the Gene Reader. As shown in FIG. 9, the nucleotide bases in green and magenta correspond to a portion of each strand that were identified as being correct and part of the (amplicon/adapter region); the nucleotide bases in red correspond to nucleotide bases that were not sequenced properly (sequencing error in that strand); and nucleotide bases in magenta represent bases that were identified as correct but part of the M13 adapter portion.

The results show that a dumbbell circular template can be constructed successfully using synthetic hairpin adapters and PCR generated amplicons. Three clones were used and each of the clones were identified during the sequencing run. The data also showed that the (+) and (−) strands can be sequenced sequentially on the same flow cell and that the localization of the sequence can be matched physically.

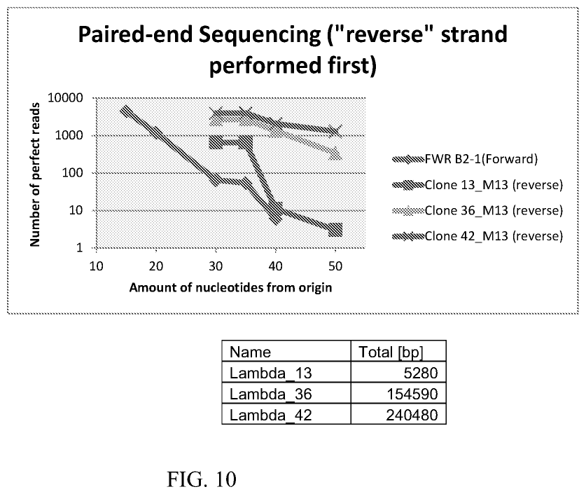

The first 50 cycles of sequential paired-end sequencing is described below and the results are shown in FIG. 10. Lambda-3 (clones 13, 36 and 42) were used. Amount of perfect reads by readlength were subdivided by clones which can be distinguished with the reverse strand sequence occurrences within the first 50 cycles (M13 adapter sequence is 27 nt). Twenty-three nt could be used for clone identification The B2 sequence is the forward strand and could not distinguish the clones for the first 50 cycles because the adapter is longer than 50 nucleotides.

By avoiding unwanted PCR primer-dimer sub-products using touchdown PCR, over 13,300 reads were mapped to the lambda clones used. Close to 1000 of these reads were perfect reads (see, Table 2, bottom panel). These results were obtained without additional condition optimization except for the PCR approach. The error rate was slightly high compared to the normal value for concatemer sequencing runs which oscillates around 6-7%.

TABLE 2

| | Base- | Mapped | Perfect | Perfect | Error |
|---|---|---|---|---|---|
| | Calls (#) | Reads (#) | Reads (#) | Reads (%) | rates (%) |
| Regular PCR | | | | | |
| (lots of | | | | | |
| primer-dimers) | | | | | |
| Forward | 6270 | 209 | 0 | 0 | 19.12 |
| Strand (+) | | | | | |
| Reverse | N/A | N/A | N/A | N/A | N/A |
| Strand (−) | | | | | |
| Forward | 57780 | 1926 | 100 | 5.19 | 13.3 |
| Strand (+) | | | | | |
| Reverse | 28469 | 630 | 0 | 0 | 12.9 |
| Strand (−) | | | | | |
| Touch- | | | | | |
| Down PCR | | | | | |
| (no primer- | | | | | |
| dimer) | | | | | |
| Forward | 400341 | 13345 | 810 | 6.07 | 12.6 |
| Strand (+) | | | | | |
| Reverse | 567016 | 13861 | 203 | 1.46 | 14.6 |
| Strand (−) | | | | | |

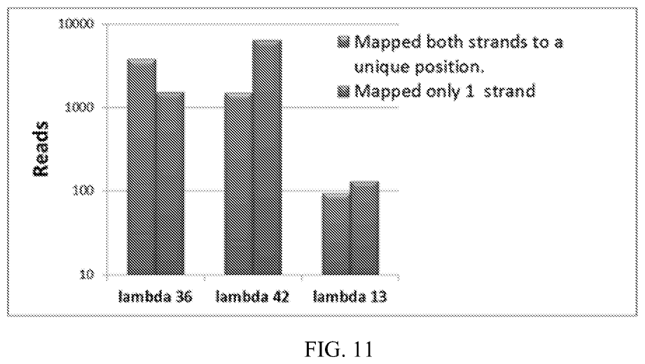

The amount of reads that can be mapped to the lambda clone sequences as well as the number of these reads that can be paired physically (originating from the same concatemer and location on the flow cell) was determined and showed that it was possible to match both strand sequences to a specific position on the flow cell (see, Table 3 and FIG. 11).

TABLE 3

| Reads | Both | One |
|---|---|---|
| lambda 36 | 3804 | 1516 |
| lambda 42 | 1489 | 6446 |
| lambda 13 | 95 | 130 |

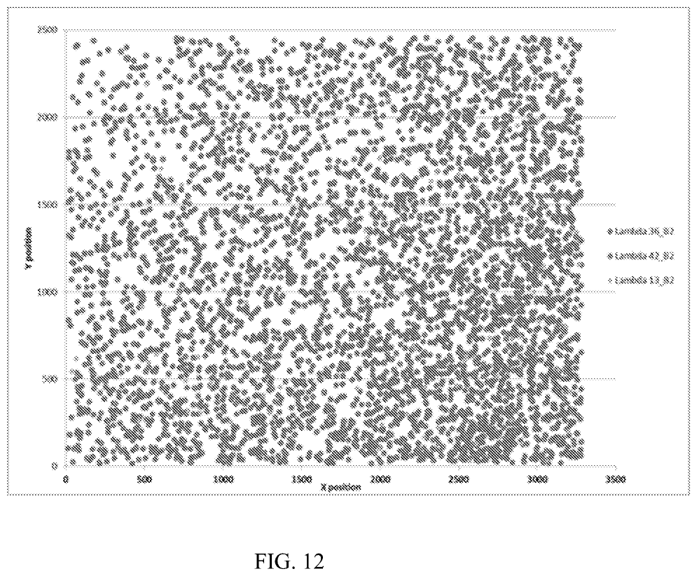

FIG. 12 shows the amount of reads that were mapped to one strand (red) or to both strands (blue) at the same time and to a unique location. The localization of the match pairs (to a unique location) for the clones 13, 36 and 42 were visualized on a flow cell. Each coordinate was color coded based on the lambda clone mapped sequence.

### Example 3: Complex Library Circular Template Preparation

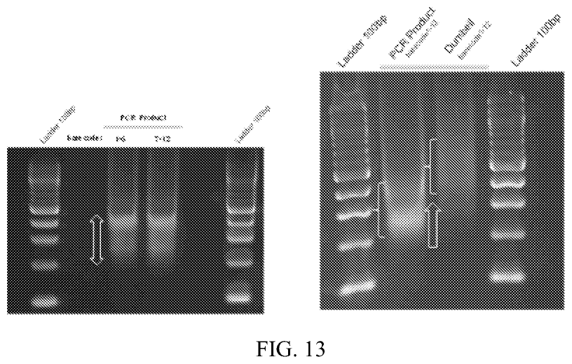

The preparation of the circular template was tested using a complex library. FIG. 13 shows the results obtained with the complex 101X library preparation showing the PCR primer products using touch-down PCR. A smear encompassing the various lengths of the amplicons was observed (braces). The products were treated with User™ to generate cohesive ends that allow the ligation of two hairpin adapters in a specific orientation. After ligation, the PCR amplicons generated circular templates as shown by an upward shift of the smear (arrows) on the gel.

### Example 4: Concatemer Crosslinking on Flow Cell

The following protocol was used to crosslink or conjugate the concatemers to the flow cell (e.g., Azigrip or PolyAn surface) using PBS alone. First, lay the empty flow cell glass-side down. Next, prepare the following solution with the rolony product (e.g., concatemer) and vortex:

| Component | Final concentration |
|---|---|
| Concatemer DNA Sample in PBS | 49 (20-100 ng/μL) |
| Reference Beads (Optional) | 1 μL (0.2 million/μL) |
| TOTAL | 50 μL |

Then, inject the solution into the flow cell and incubate at room temperature for two hours. This is followed by washing the flow cell with 1 mL 1× with enzymology buffer (wash buffer B [9/10]). Then, prepare the following solution and vortex:

| Component | Final concentration |
|---|---|
| Buffer A | 3 μL |
| FWR Seq Primer (250 μM) | 0.6 μL of 250 μM |
| H2O | 26.4 μL H2O |
| TOTAL | 30 μL |

Next, aspirate the wash buffer B and inject the solution into the flow cell. Wipe the back of the flow cell with a kimwipe and tape ports closed with clear tape. Heat the flow cell to 65° C. for 10 minutes, and move the flow cell to the bench top to cool to room temperature for 10 minutes. Alternatively, heat the flow cell at 90° C. for 5 minutes and gradually anneal to room temperature by switching off the heat block. Proceed to GeneReader™ sequencing or single base extension. Repeat the steps beginning with incubating at room temperature for two hours for paired-end sequencing by replacing the forward sequencing primer with the reverse sequencing primer.

Next, the concatemers generated by a circle formed by the ligation of two hairpin structures to a PCR amplicon are sequenced. The sequencing can be performed in both directions subsequently.

### Example 5: Concatemer Conjugation/Crosslinking onto Flow Cell Using EDC, NHS, and BS3

The following protocol was used to crosslink the concatemers to a flow cell (e.g., Azigrip or PolyAn surface). Next, the concatemers generated by a circle formed by the ligation of two hairpins were sequenced. The sequencing can be performed in both directions subsequently.

First, lay the empty flow cell glass-side down. Next prepare the following solution with the rolony product (e.g., concatemer) and vortex:

| Component | Final concentration |
|---|---|
| Rolony DNA Sample | 43.5 (50-100 ng/μL) |
| Reference Beads (Optional) | 0.2 million/μL |
| 1-ethyl-3-(3-dimethylaminopropyl) | 2.5 μL of 300 mg/mL |
| carbodiimide hydrochloride (EDC) | |
| N-Hydroxy Succinimide (NHS) | 2.5 μL of 150 mg/mL |
| Bis-SulfoSuccinimidyl Suberate (BS3) | 1.3 μL of 2 mg pellet/140 μL |
| | 50 μL |

Next, inject the solution into the flow cell and incubate at room temperature for two hours. This is followed by washing the flow cell with 1 mL 1× with enzymology buffer (wash buffer B). Then, prepare the following solution and vortex:

| Component | Final concentration |
|---|---|
| Buffer A | 3 μL |
| FWR Seq Primer (250 μM) | 0.6 μL of 250 μM |
| H2O | 26.4 μL H2O |
| TOTAL | 30 μL |

Next, aspirate the wash buffer B and inject the solution into the flow cell. Wipe the back of the flow cell with a kimwipe and tape ports closed with clear tape. Heat the flow cell to 65° C. for 10 minutes, and move the flow cell to the bench top to cool to room temperature for 10 minutes. Alternatively, heat the flow cell at 90° C. for 5 minutes and gradually anneal to room temperature by switching off the heat block. Proceed to GeneReader™ sequencing or single base extension. Repeat the steps beginning with incubating at room temperature for two hours for paired-end sequencing by replacing the forward sequencing primer with the reverse sequencing primer.

### Example 6: Rolling Circle Amplification of Concatemers

The following experiment was performed to test the approach wherein two hairpin structures are ligated to a PCR amplicon using T4 DNA ligase to form a circle.

RecA was used: final concentration, 0.08 μg/μL; and stock concentration, 2 μg/μL.

The following protocol was used for rolling circle amplification:

| | Volume | |
|---|---|---|
| Component | [μl] | Final concentration |
| Nuclease free water | 76 | |
| Enzymatics 10× reaction buffer | 10 | 1× (Enzymatic) |
| 1 mM AA-dUTP | 1 | 10 μM |
| 10 mM dNTP mix | 4 | 1 mM each |
| Rolony primer (2 μM) | 1 | 0.02 μM |
| Circularized DNA (20 ng/μL) | x | Lambda total 1 pmol |
| RecA | 8 μL | 0.08 ug/μL |
| | 100 μl | |

Qubit was used to measure the concentration of the circulized DNA. For RecA and control, the following steps were carried out: 1) anneal for 2 minutes at 95° C.; slow cool to 30° C. (Duplex program) and pause at 4° C. before going to next step; 2) add 1 μl of Phi 29 DNA polymerase (Enzymatics or NEB) while reaction tube is in thermocycler at 4° C. or on ice; 3) incubate at 30° C. for 6 hrs; 4) stop reaction by adding 300 μL of 1×PBS; 5) store at 4° C.; and 6) measure DNA concentration with Qubit ssDNA kit, use 1 μL directly.

### Example 7: Conjugation/Crosslinking of Concatemers onto Flow Cell Using EDC, NHS, and BS3

The purpose of the next experiment was to sequence the concatemers generated by a circle formed by the ligation of twohairpins. Sequencing was performed in both directions on the same flow cell.

The following protocol was used to crosslink the concatemers onto a flow cell. First, lay the empty flow cell glass-side down. Next, prepare following solution with rolony product (e.g., concatemer) and vortex:

| Component | Final conc |
|---|---|
| Rolony DNA Sample | 43.5 (50-100 ng/μL) |
| Reference Beads (Optional) | 0.2 million/μL |
| 1-ethyl-3-(3-dimethylaminopropyl) | 2.5 μL of 300 mg/mL |
| carbodiimide hydrochloride (EDC) | |
| N-Hydroxy Succinimide (NHS) | 2.5 μL of 150 mg/mL |
| Bis-SulfoSuccinimidyl Suberate (BS3) | 1.3 μL of 2 mg pellet/140 μL |
| | 50 μL |

Next, inject the solution into the flow cell and incubate at room temperature for 2 hours. The flow cell is then washed with 1 mL 1× enzymology buffer (wash buffer B [9/10]). The following solution is prepared and vortexed:

| | Final |
|---|---|
| Component | concentration |
| Buffer A | 3 μL |
| FWR or REV Seq Primer (100 μM) | 1.5 μL of 250 μM |
| H2O | 25.5 μL H2O |
| TOTAL | 30 μL |

Aspirate wash buffer B and inject solution into the flow cell. Wipe the back of flow cell with kimwipe and tape the ports closed with clear tape. Heat the flow cell to 65° C. for 10 minutes and then move the flow cell to the bench top to cool to room temperature for 10 minutes. Alternatively, heat the flow cell to 90° C. for 5 min and gradually anneal to room temperature by switching off the heat block. Proceed to GeneReader™ sequencing or single base extension.

Before proceeding with the reverse paired-end reverse strand, a blocking step is required. For this, the blocking mixture is prepared according to Table 4:

TABLE 4
Blocking Mixture.

| Reagent | Volume | Final conc. |
|---|---|---|
| 10 mM ddATP | 0.6 μL | 200 μM |
| 10 mM ddGTP | 0.6 μL | 200 μM |
| 10 mM ddCTP | 0.6 μL | 200 μM |
| 10 mM ddTTP | 0.6 μL | 200 μM |
| Pol Extend | 3 μL | |
| Extend Premix A1 | 24.6 μL | |
| Total | 30 μL | |

*The volume is set for total 30 μL (the protocol can be adjusted, for example, to prepare a solution for the equivalent of two flow cells (60 μL)).

For the reverse paired-end reverse strand portion of the experiment, wash the flow cell with wash buffer 9/10 (2×200 μL), and add 30 μL to the flow cell, incubating at 65° C. for 30 minutes. Pipette 1 mL of wash buffer B into each flow cell, then aspirate (5×200 μL). The flow cell(s) are now ready for reverse primer hybridization. Repeat the steps (e.g., aspirate wash buffer B and inject solution into the flow cell; wipe the back of flow cell with kimwipe and tape the ports closed with clear tape; heat the flow cell to 65° C. for 10 minutes and then move the flow cell to the bench top to cool to room temperature for 10 minutes (alternatively, heat the flow cell to 90° C. for 5 min and gradually anneal to room temperature by switching off the heat block); and proceed to GeneReader™ sequencing or single base extension) for paired-end sequencing by replacing the forward sequencing primer with the reverse sequencing primer.

### Example 8: Using the Circular Templates I

The purpose of this experiment was to ligate the hairpin adapters to clones 13, 36 and 42 with cohesive ends. Clones were generated using uracil-containing primers. The approach involved conducting a ligation reaction individually and with both end-hairpin adapters simultaneously to generate large closed circles (e.g., in the shape of a dumbbell).

The following materials were used: clones 13, 36, and 42 and double-stranded ligation product (GeneWiz); T4 ligase buffer (NEB), T4 ligase enzyme (NEB), 2.2% agarose Lonza flash gel (Lonza) and Qiaquick PCR purification kit (Qiagen).

The ligation protocol included the following components: User™-treated clones 13, 36 and 42; forward hairpin adapter, 1:10 dilution (about 20 ng/μL); and reverse hairpin adapter; 1:10 dilution (about 20 ng/4). Each ligation reaction contained 20 ng of amplicon DNA and maintained an amplicon/hairpin ratio of 1:4. The following components were combined: amplicon DNA (20 ng) and 4 μl (measured by Q-bit) (92400 g/mol; 20 ng=2.16E-13 mol); hairpin DNA (4-fold molar excess)˜18,810 g/mol (8.66E-13 mol). A total of 1.63E-08 g was used. A solution comprising the following components was prepared: reaction buffer, 10×5 μl, water 21.7 μl, and T4 DNA Ligase 1 μl (1 unit), for a total of 30 μl.

The components and solution were mixed and incubated at 16° C. for 12 hours or at room temperature for 2 hours, and then stored at −20° C. or purified using Qiaquick. Concentrations were measured using Qubit.

### Example 9: Using the Circulating Templates II

Three clones from a lambda library were used to generate three distinct circular templates that can be sequenced. The three clones lambda were used in a PCR recation with PCR uracil containing primers. After USER enzyme treatment the cohesive ends generated can be ligated to hairpin adapters

The following materials were used: clones 13, 36, and 42 and double-stranded ligation product (GeneWiz); Cutsmart buffer (B7204S; NEB), User™ enzyme (M5505S; NEB), 2.2% agarose Lonza flash gel (Lonza) and Qiaquick PCR purification kit (Qiagen).

For this experiment, USER™ enzyme was used to nick the duplex and create cohesive ends: nick a 100 pmol aliquot of the library. The following components were used:

| Component | |
|---|---|
| Clone 36 library (10 ng/uL) | 10 μL (160 fmol) |
| 10× Cutsmart buffer | 1.5 μL |
| USER enzyme mix (1 unit/uL) | 1 μL (1 units/10 pmol) |
| Nuclease-free water | 7.5 μL |
| Total volume | 20 μL |

The following steps were carried out: 1) incubate for 45 min at 37° C.; 2) purify with Qiaquick PCR purification kit and elute in 30 μL EB buffer; and 3) quantify with Qubit (High Sensitivity).

### Example 10: PCR Optimization for Lambda Clones

This experiment reduced primer-dimers that generate smaller circles using circligase and guide-DNA or hairpin-ligation (forming a circular template that has the shape of a dumbbell).

The PCR components used are as follows:

| | |
|---|---|
| Uracil-containing B2 fwd (10 μM) | 2.5 μl |
| Uracil-containing M13 (10 μM) M13 | 2.5 μl |
| 10× Hot Start Taq Reaction Buffer | 5 μl |
| 10 mM dNTPs | 1 μl |
| Lambda Clones #13, 36, 42, (separately) | 1 ng (1 μl) |
| Hot Start Taq DNA Polymerase (2 U/μl) | 0.5 μl |
| Nuclease Free H2O | 37.5 μl |

The Touchdown PCR conditions are as follows:
95° C. 4 min;
94° C. 1 min;
75° C. 1 min; −1° C. each cycle 15×;
68° C. 2 min
94° C. 30 sec;
57° C. 30 sec 30×;
72° C. 1 min;
72° C. 5 min; and
4° C.—end.

### Example 11: Protocol for Seeding and Crosslinking Concatemers on a Flow Cell and Sequencing Primer Hybridization onto the Concatemers

Concatemer seeding on azigrip flow cell using the following seeding solution:

| Concatemer Seeding | | Final | |
|---|---|---|---|
| Solution | Volume | Concentration | |
| Undiluted Concatemers Stock | 3 μl | ~15 | ng/μl |
| Buffer (e.g., 1× PBS) | 27 μl | | |
| Reference Bead | (0.3 μl) | 0.01 | million/μl |

| | |
|---|---|
| (1M/μl, optional) | |
| Total Volume | 30 μl |

The concatmer concentrations used were at 100-150 ng/μL for current working stock. A 1/10 dilution was used (e.g., ˜500 ng total). More diluted solutions can be used. For example, a final concentration of 43 ng/4 and a volume of 10.5 μL was used.

The following protocol was carried out: 1) prepare concatemer seeding solution as described above; 2) inject 30 μl of the concatemer seeding solution into flow cell; and 3) incubate for 2 hr at room temperature.

For crosslinking rolonies (e.g., concatemers) onto a flow cell, the following components were used:

| Crosslinking Chemistry | Volume |
|---|---|
| 1-ethy1-3-(3-dimethylaminopropyl) | 7.5 μl of 300 mg/mL |
| carbodiimide hydrochloride (EDC) | |
| N-Hydroxy Succinimide (NHS) | 7.5 μl of 150 mg/mL |
| Bis-SulfoSuccinimidyl Suberate | 3.9 μl of 2 mg |
| (BS3) | pellet/140 μl |
| 1× PBS | 131.1 μl |
| Total Volume | 150 μl |

If crosslinking is required, wash the flow cell with 3×200 μl 1×PBS. Next, inject 150 μl of the crosslinking chemistry into the flow cell and incubate for 30 min.

For the sequencing primer hybridization step, the following solution was used:

| Sequencing Primer Solution | Final volume |
|---|---|
| Buffer A | 15 μl |
| Seq Primer (250 uM) | 3 μl of 250 μM |
| H2O | 132 μl H2O |
| TOTAL | 150 μl |

For this portion of the experiment, the following protocol was used: 1) wash the flow cell with 3×200 μl wash buffer B; 2) Inject 150 μl of the sequencing primer into the flow cell; 3) tape the flow cell ports and incubate at 65° C. for 10 min; 4) remove the flow cell from heat and allow it to cool to room temperature for 10 min; and proceed to sequencing.

### Example 12: Paired-End Approach (Sequential on Flow Cell)

Second strand amplification of the concatemers was performed on a flow cell. The second amplification reaction was followed by a block with ddNTP and sequencing using B2 reversesequencing primer (e.g., 41mer).

The reaction protocol is described below. The second strand sequencing reaction is as follows: 1) control flow cells were used (with 3 chemistry X-link and 1×PBS); 2) wash the flow cell with 200 μL of PBS into channel three times (slowly); and 3) aspirate each flow cell. Next, add 50 μL of amplification/Elongation mix (see below) to each flow cell for the second strand elongation. Incubate at 30° C. for 4 hours. Then, pipette 1 mL of wash buffer 9/10 into each flow cell, followed by aspiration (e.g., 5λ200 μL).

| | Volume |
|---|---|
| Component | (μL) |
| H2O | 167 |
| Enzymatics 10× reaction buffer | 20 |
| Enzymatics Phi29 Polymerase | 2 |
| dNTP mix | 8 |
| AA-dUTP | 2 |
| B2 primer (2 uM) | 1 |
| Total | 200 |

For the blocking step, add the following solution, incubate at 30° C. for 1 hour followed by piptetting 1 mL of wash buffer 9/10 into each flow cell. Then, aspirate (e.g., 5×200 μL).

| | Volume |
|---|---|
| Component | (μL) |
| H2O | 168 |
| Enzymatics 10× reaction buffer | 20 |
| Enzymatics Phi29 Polymerase | 2 |
| ddNTP mix (1 mM) | 8 |
| AA-dUTP | 2 |
| TOTAL | 200 |

Next, prepare the following primer solution for sequencing:

| | Final |
|---|---|
| Component | concentration |
| Buffer A | 3 μL |
| Seq Primer (Reverse B2; 41 mer) | 2.5 μL of 100 μM |
| (100 μM) | |
| H2O | 26.4 μL H2O |
| TOTAL | 30 μL |

The following protocol was carried out: 1) aspirate the wash buffer B; 2) inject the solution into the flow cell; 3) wipe the back of flow cell with a kimwipe and tape ports closed with clear tape; 4) heat to 65° C. for 10 minutes, then move the flow cell to the bench top to cool to room temperature for 10 minutes; and 5) proceed to GeneReader™ sequencing.

1. A method of preparing a plurality of concatamers, comprising:
a. forming a first partially double stranded circular DNA wherein the partially double stranded circular DNA contains (i) a sequence of interest having a first strand and a second strand, (ii) a first hairpin adaptor, and (iii) a second hairpin adaptor, wherein the first and second strands of the sequence of interest are complementary to each other and wherein the first hairpin adaptor comprises a first primer binding site and the second hairpin adaptor comprises a second primer binding site, wherein the first hairpin adaptor and the second hairpin adaptor are different; and
b. amplifying the first partially double stranded circular DNA via rolling circle amplification, wherein amplification of the first partially double stranded circular DNA results in replicated strands, wherein during amplification at least one of the replicated strands is displaced from the first partially double stranded circular DNA by strand displacement replication;
wherein the amplification of the first partially double stranded circular DNA results in a plurality of concatemers.

2. (canceled)

3. The method of claim 1, wherein the first partially double stranded circular DNA is in a dumbbell configuration having a double stranded portion flanked by two single stranded portions.

4. (canceled)

5. The method of claim 3, wherein one of the two single stranded portions comprises part of the first hairpin adaptor and one of the two single stranded portions comprises the second hairpin adaptor.

6. The method of claim 1, wherein each of the concatamers comprises a single stranded DNA portion and a double stranded DNA portion.

7. (canceled)

8. The method of claim 1, wherein each of the concatamers comprises a copy of the sequence of interest and a copy of at least one of the hairpin adaptors.

9. (canceled)

10. The method of claim 1, wherein the concatemers comprise, in order, a portion of one of the hairpin adaptors, a copy of the first strand of the sequence of interest, a copy of one of the hairpin adaptors, and a copy of the second strand of the sequence of interest, wherein the first and second strands of the sequence of interest are hybridized together.

11.-15. (canceled)

16. The method of claim 1, wherein the first hairpin adapter comprises a first restriction enzyme binding site.

17. The method of claim 1, wherein the first hairpin adapter is partially single-stranded, wherein the single-stranded portion comprises an overhang portion complementary to one terminus of the sequence of interest.

18. The method of claim 1, wherein the second hairpin adapter comprise a second restriction enzyme binding site.

19. (canceled)

20. The method of claim 1, further comprising immobilizing the plurality of concatemers on a surface of a substrate.

21.-27. (canceled)

28. The method of claim 1, wherein step b) comprises amplifying the first partially double stranded circular DNA modified nucleotides in the presence of modified nucleotides.

29. The method of claim 28, wherein the modified nucleotides comprise a bromide or thiol group.

30. (canceled)

31. The method of claim 1, wherein the first partially double-stranded circular DNA is formed by:
i. contacting a target nucleic acid molecule that contains a sequence of interest, with a first primer set, wherein at least one primer in the first primer set comprises a uracil at the 3′ end;
ii. incubating the target nucleic acid molecule with the first primer set under conditions that promote hybridization and replication of the target nucleic acid molecule, thereby producing a double-stranded amplicon, wherein the double-stranded amplicon comprises a first and second strand;
iii. contacting the double-stranded amplicon with an enzyme to produce a first terminal overhang at the 3′ end of the first strand and a second terminal overhang at the 3′ end of the second strand of the double-stranded amplicon;
iv. contacting the double-stranded amplicon of iii) with the first hairpin adaptor and the second hairpin adaptor, wherein the first hairpin adaptor comprises a first sequence complementary to the first terminal overhang of the double-stranded amplicon and the second hairpin adapter comprises a second sequence complementary to the second terminal overhang;
v. incubating the double-stranded amplicon with the first hairpin adaptor and the second hairpin adapter under conditions that promote hybridization between the first hairpin adapter with the first terminal overhang of the double-stranded amplicon and the second hairpin adapter with the second terminal overhang of the double-stranded amplicon; and
vi. ligating the first hairpin adaptor to the first terminal overhang and the second hairpin adaptor with the second terminal overhang thereby forming the first partially double stranded circular DNA.

32.-44. (canceled)

45. An array for identifying at least one nucleotide of a sequence of interest, comprising a plurality of amplicons immobilized on a surface, wherein each of the amplicons comprise two or more concatemers, wherein each of the concatemers comprises: a first hairpin adapter, at least one sequence of interest and a second hairpin adapter formed by a process comprising:
a) contacting a target nucleic acid molecule comprising the sequence of interest, with a first primer set having a uracil at the 3′ end, wherein the target nucleic acid molecule is double-stranded; incubating the target nucleic acid molecule with a first primer set under conditions that promote hybridization and replication of the target nucleic acid molecule, thereby producing a double-stranded template, wherein the double-stranded template comprises a first and second strand;
b) contacting the double-stranded template with an enzyme to produce a first terminal overhang and a second terminal overhang at the 3′ end of the first and second strand of the double-stranded template;
c) contacting the double-stranded template with the first hairpin adaptor and second hairpin adaptor, wherein the first hairpin adaptor comprises a first sequence complementary to the first terminal overhang the double-stranded template and the second hairpin adapter comprises a second sequence complementary to the second terminal overhang at the 3′ end of the double-stranded template;
d) incubating the double-stranded template with the first hairpin adaptor and second hairpin adapter under conditions that promote hybridization between the first hairpin adapter with the first terminal overhang of the double-stranded template and the second hairpin adapter with the second terminal overhang of the double-stranded template; and
e) ligating the first hairpin adaptor to the first terminal overhang and the second hairpin adaptor with the second terminal overhang forming a partially double stranded circular DNA;
wherein the first adaptor is different from the second adaptor and comprises a first restriction enzyme binding site and the second adaptor comprises a second restriction enzyme binding site,
wherein the first and second restriction enzyme binding sites that cleaves DNA at cleavage site.

46.-71. (canceled)

72. A method of identifying at least one nucleotide of a sequence of interest, the method comprising:
a) contacting at least one partially double stranded concatamer with a first primer, wherein the first primer hybridizes to a first primer binding site,
b) extending the first primer in the presence of one or more nucleotide analogues that comprise a 3′OH-protecting group thereby generating a first primer elongation product,
c) contacting at least one of the partially double stranded concatamers with a second primer, wherein the second primer hybridizes to a second primer binding site;
d) extending the second primer, thereby generating a second primer elongation product; and
e) identifying at least one nucleotide of the sequence of interest adjacent or close to the first primer binding site and at least one nucleotide of the sequence of interest adjacent or close to the second primer binding site.

73. The method of claim 72, wherein prior to step b) the first primer is extended in the absence of one or more nucleotide analogues.

74. (canceled)

75. (canceled)

76. The method of claim 73, wherein the one or more nucleotide analogues are added after the first primer is extended in the absence of one or more dideoxynucleotides.

77. (canceled)

78. The method of claim 72, wherein the partially double stranded concatamer comprises a single stranded DNA portion and a double stranded DNA portion, wherein the first and second primer binding sites are located in a single stranded region of the partially double stranded concatamer, and wherein the first and second primer binding sites are different.

79.-88. (canceled)

89. The method of claim 72, wherein the 3′OH-protecting group of the one or more nucleotide analogues is a reversible 3′OH-protecting group.

90. The method of claim 72, wherein the 3′OH-protecting group of the one or more nucleotide analogues is an irreversible 3′OH-protecting group.

91. The method of claim 72, wherein the one or more nucleotide analogues further comprise a unique label attached through a cleavable linker attached to the base of the nucleotide analogues.

92.-107. (canceled)
